(19) United States
(12) Patent Application Publication
(10) Pub. No.: US 2021/0275433 A1
(43) Pub. Date: Sep. 9, 2021

(54) JANUS PARTICLES AND THEIR USE FOR SURFACTANT-FREE CLEANSING AND EMULSION STABILIZATION

(71) Applicant: The Trustees of Princeton University, Princeton, NJ (US)

(72) Inventors: Rodney D. PRIESTLEY, Princeton, NJ (US); Robert K. PRUD'HOMME, Princeton, NJ (US); Sunny NIU, Princeton, NJ (US)

(73) Assignee: The Trustees of Princeton University, Princetion, NJ (US)

(21) Appl. No.: 16/338,894

(22) PCT Filed: Oct. 2, 2017
(86) PCT No.: PCT/US2017/054779

§ 371 Date: Apr. 2, 2019

Related U.S. Application Data
(60) Provisional application No. 62/403,619, filed on Oct. 3, 2016.
(60) Provisional application No. 62/403,625, filed on Oct. 3, 2016.

(51) Int. Cl. A61K 8/90; A61Q 19/10; A61K 8/04; A61K 8/06; A61K 8/85; B01J 13/00

(52) U.S. Cl. CPC A61K 8/90; A61Q 19/10; A61K 8/044; A61K 8/062; A61K 2800/412; A61K 8/85; B01J 13/0021; A61K 2800/33; A61K 2800/654; A61K 8/068

(57) ABSTRACT

Janus particles, including biodegradable, biocompatible, anisotropic, amphiphilic Janus nanocolloids, and their use in stabilizing emulsions and cleansing are described.

The present invention relates to polymer Janus particles, including biodegradable and/or biocompatible asymmetric Janus nanocolloids, geometrical Janus micelles and processes of making them, emulsions stabilized by Janus nanoparticles, and methods of using Janus particles for surfactant-free cleansing. This application claims the benefit of U.S. Provisional Application No. 62/403,619, filed Oct. 3, 2016, and U.S. Provisional Application No. 62/403,625, filed Oct. 3, 2016, which are hereby incorporated by reference in their entirety.

### FIELD OF THE INVENTION

### Background of the Invention

In Pierre Gilles de Gennes's 1991 Nobel Laureate speech titled “Soft Matter” he introduced the concept of Janus particles, which are anisotropically structured particles containing two distinct regions of material or functionality. Their development can be considered in the context of the scientific and technological development of other chemically anisotopically structured materials, such as surfactants and block copolymers. The ability to synthesize surfactants at scale and in cost effective ways has led to the current surfactant market. The ability to synthesize block copolymers at scale and in cost effective ways has led to the current market for thermoplastic elastomers based on block copolymers.

Janus colloids can be assembled from a broad variety of building blocks ranging from metals to polymers (Yoon, J. et al., Amphiphilic colloidal surfactants based on electrohydrodynamic co-jetting, ACS Appl. Mater. Interfaces, 2013, 5, 11281-7; Glaser, N. et al., Janus particles at liquid-liquid interfaces, Langmuir 2006, 22, 5227-9). The breadth of material properties exhibited by polymers as well as their ability to phase separate can be useful for the generation of Janus colloids.

Colloids possessing patterned or structured surface domains of differing chemical composition can serve as nanoscale building blocks for the design of materials with molecular scale features (Walther, A. et al., Janus particles. Soft Matter 2008, 4, 663-668; Walther, A. et al. Janus Particles: Synthesis, Self-Assembly, Physical Properties, and Applications, Chem. Rev. 2013, 113, 5194-5261; Samuel, A. Z. et al., Self-Adapting Amphiphilic Hyperbranched Polymers, Macromolecules 2012, 45, 2348-2358). The functionality of such particles can depend on the spatial topology and molecular properties of surface domains (Walther, A. et al., Janus particles, Soft Matter 2008, 4, 663-668; Walther, A. et al., Janus Particles: Synthesis, Self-Assembly, Physical Properties, and Applications, Chem. Rev. 2013, 113, 5194-5261). Janus nanocolloids can assemble into higher-order superstructures when induced to by various environmental stimuli (Walther, A. et al., Janus particles, Soft Matter 2008, 4, 663-668; Chen, Q. et al., Directed self-assembly of a colloidal kagome lattice, Nature 2011, 469, 381-384). They can, for example, organize under magnetic (Smoukov, S. K. et al., Reconfigurable responsive structures assembled from magnetic Janus particles, Soft Matter 2009, 5, 1285-1292) or electric fields (Gangwal, S. et al., Dielectrophoretic Assembly of Metallodielectric Janus Particles in AC Electric Fields, Langmuir 2008, 24, 13312-13320) to form patterned chains on solid substrates, undergo complex translational (Gangwal, S. et al., Induced-Charge Electrophoresis of Metallodielectric Particles, Phys. Rev. Lett. 2008, 100, 058302) and rotational (Gangwal, S. et al. Induced-Charge Electrophoresis of Metallodielectric Particles, Phys. Rev. Lett. 2008, 100, 058302) motion in alternating fields (Squires, T. M. et al., Breaking symmetries in induced-charge electro-osmosis and electrophoresis, J. Fluid Mech. 2006, 560, 65-101), migrate to the interface between two immiscible fluids in order to decrease the surface tension of macroscopic emulsions (Yoon, J. et al., Amphiphilic colloidal surfactants based on electrohydrodynamic co-jetting, ACS Appl. Mater. Interfaces 2013, 5, 11281-7), and uniquely interact with cellular interfaces in order to facilitate the absorption of imaging or therapeutic agents (Gao, Y. et al., How half-coated janus particles enter cells, J. Am. Chem. Soc. 2013, 135, 19091-4).

The interest in multi-faced nanocolloid applications has outstripped the ability to produce commercial-scale materials, hindering the development of new technologies (Samuel, A. Z. et al., Self-Adapting Amphiphilic Hyperbranched Polymers, Macromolecules 2012, 45, 2348-2358; Jang, S. G. et al., Striped, ellipsoidal particles by controlled assembly of diblock copolymers, J. Am. Chem. Soc. 2013, 135, 6649-57; Erhardt, R. et al., Amphiphilic Janus micelles with polystyrene and poly(methacrylic acid) hemispheres, J. Am. Chem. Soc. 2003, 125, 3260-3267; Roh, K. et al., Biphasic Janus particles with nanoscale anisotropy, Nat. Mater. 2005, 4, 759-763; Yamashita, N. et al., Preparation of hemispherical particles by cleavage of micrometer-sized, spherical poly(methyl methacrylate)/polystyrene composite particle with Janus structure: effect of molecular weight, Colloid Polym. Sci. 2013, 292, 733-738).

The scalability of processes for forming and comprehensive control over particle morphology of Janus particles is a challenge (Chang, E. P. et al., Membrane Emulsification and Solvent Pervaporation Processes for the Continuous Synthesis of Functional Magnetic and Janus Nanobeads, Langmuir 2012, 28, 9748-9758; Wang, Y. et al., Colloids with valence and specific directional bonding, Nature 2012, 491, 51-5; Walther, A. et al., Janus discs, J Am. Chem. Soc., 2007, 129, 6187-98).

Emulsions are dispersions of one immiscible liquid phase in another, stabilized by an interfacially active molecule or particle (Becher, P.; Fishman & M. M. Tech. Emuls. 1965, 267-325). They are utilized in a number of practically significant commercial products, such as in pharmaceuticals for increased biocompatibility and improved drug distribution (Yeeprae, W. K. et al. J. Drug Target. 2005, 13 (8-9), 479-487. Collins-Gold, L. C. et al. Advanced Drug Delivery Reviews. 1990, 189-208. Rawat, M. et al. Yakugaku Zasshi 2008, 128 (2), 269-280.), cosmetics for enhanced tactual and biophysical properties (Sonneville-Aubrun, O. et al. Adv. Colloid Interface Sci. 2004, 108-109, 145-149. Miller, D. J. et al. Colloids Surfaces A Physicochem. Eng. Asp. 2001, 183-185, 681-688.), food for prolonged stability and shelf life (Dickinson, E. Current Opinion in Colloid and Interface Science. 2010, 40-49.), and drilling fluids for enhanced oil extraction (Caenn, R. & Chillingar, G. V. Pet. Sci. Eng. 1996, 14, 221-230.). Emulsion-based products must have sufficient shelf-life, the dispersed liquid droplets of the emulsion must resist fusion and separation into two bulk phases. Various interfacial stabilizing agents can improve the kinetic stability of the emulsions by reducing the interfacial tension between the two liquid phases and mitigating droplet coalescence (Baret, J. C. et al. Langmuir 2009, 25 (11), 6088-6093.). Homogenized milk, for example, contains β-casein and β-lactoglobulin proteins that adsorb onto the interface between fat globules and the bulk aqueous phase, prolonging the stability of the milk emulsion by creating a low energy, amphiphilic boundary between the oil and water phases (Dickinson, E. Colloids Surfaces B Biointerfaces 2001, 20, 197-210.). Low molecular weight surfactants (Baret et al. 2009) and surface-active polymers (Riess, G. et al. Polym. Eng. Sci. 1977, 17 (8), 634-638. Saigal, T. et al. J. Colloid Interface Sci. 2013, 394 (1), 284-292.) are used as emulsifiers. However, these stabilizing agents can be unsuitable for certain applications due to their small size and lack of biocompatibility. For example, some surfactants are skin irritants, so that they cannot be used for cosmetic or dermatological products (Effendy, I. & Maibach, H. I. Contact Dermatitis. 1995, pp 217-225.). Moreover, the disposal of surfactants used in household and industrial products into aquatic and terrestrial environments can have detrimental effects on wildlife. Some small molecule surfactants may have a negative impact on human health (Jobling, S. et al. Environ. Sci. Technol. 1998, 32 (17), 2498-2506. Ying, G. G. Environment International. 2006, 417-431.).

### SUMMARY

In an embodiment of the invention, an emulsion includes a asymmetric Janus (two-faced) nanoparticle (a nanocolloid) that has a hydrophilic surface as a hydrophilic face and a hydrophobic surface as a hydrophobic face, a hydrophilic liquid (such as water), and a hydrophobic liquid (such as an aliphatic hydrocarbon oil), and includes an interface between the hydrophilic liquid and the hydrophobic liquid, with the asymmetric Janus nanocolloid located at the interface. The Janus nanocolloid can be formed of at least one organic polymer.

In an embodiment, the Janus (two-faced) nanocolloid includes a block copolymer that has a more hydrophilic block and a more hydrophobic block. The Janus nanocolloid can further include a homopolymer. The block copolymer and/or the homopolymer can be biodegradable and/or biocompatible.

In an embodiment, the Janus (two-faced) nanocolloid includes polylactic acid (PLA) polymer and polyethylene oxide-block-polycaprolactone (PEO-b-PCL) copolymer.

In an embodiment of the invention, an asymmetric Janus nanocolloid includes a first hydrophobic polymer, a second polymer, and an amphiphilic block copolymer. The asymmetric Janus nanocolloid can have a surface with a hydrophobic face at the surface and a hydrophilic face at the surface. The first hydrophobic polymer can form the hydrophobic face, and the second polymer can form the hydrophilic face. The second polymer can have amine or carboxylic functionality at its terminal end. The amphiphilic block copolymer can include a hydrophobic block formed of the same monomer units of which the first hydrophobic polymer is formed. Polyethylene glycol polymers can be coupled through 1-ethyl-3-(3-dimethylaminopropyl)carbodiimide (EDC) molecules to the amine functionality of the second polymer. An emulsion can include a hydrophilic liquid (for example, water), a hydrophobic liquid (for example, an oil, including a naturally occurring oil, such as sebum, which can include some or all of a triglyceride, a wax ester, squalene, a free fatty acid, and sapienic acid), and an asymmetric Janus nanocolloid.

In an embodiment of the invention, an asymmetric Janus nanoparticle includes a high glass transition temperature (Tg) polymer and a low glass transition temperature (Tg) polymer. The high glass transition temperature polymer can have hydrophilic groups at a hydrophilic face of the Janus nanoparticle, and the low glass transition polymer can have oleophilic groups at an oleophilic face of the Janus nanoparticle. The high glass transition temperature polymer can be polystyrene functionalized with hydrophilic groups, and the low glass transition temperature polymer can be polyisoprene.

A method according to the invention is for cleansing skin or another surface and includes applying a colloid suspension comprising the asymmetric Janus nanoparticle and water to the skin or the other surface, and rinsing the colloid suspension from the skin or other surface with water.

In an embodiment of the invention, a geometrical Janus micelle includes an aggregate of a more than one Janus particle, wherein each Janus particle has a surface having a hydrophilic face and an oleophilic face. The oleophilic face of each Janus particle can be oriented toward a center of mass of the aggregate.

A method according to the invention is for forming a geometrical Janus micelle and includes forming Janus particles in a first flash nanoprecipitation step, each Janus particle including a surface including a hydrophilic face and an oleophilic face, suspending the Janus particles in a process solvent to form a process solution, and continuously mixing the process solution with a nonprocess salt solution in a second flash nanoprecipitation step to form the geometrical Janus micelle.

### BRIEF DESCRIPTION OF THE FIGURES

FIG. 1 shows a schematic of the Flash NanoPrecipitation (FNP) method.

FIGS. 2A and 2B show tunneling electron microscopy (TEM) images of the nanoparticles formed as the overall feed concentration and ratio of polystyrene (PS) and polyisoprene (PI) polymers is varied. FIG. 2A represents nanoparticles formed with lower molecular weight PS and PI polymers, and FIG. 2B represents nanoparticles formed with higher molecular weight PS and PI polymers. FIG. 2C indicates the morphology (Janus or multi-faced) of the nanoparticles formed with respect to the PS/PI ratio and the dimensionless particle diameter along with a comparison to scaling theory.

FIG. 3 shows the Di10, Di50, and Di90 particle diameter values and the Span, indicating particle size distribution, for particles formed as a function of overall feed concentration with a 50:50 mass ratio of polystyrene (PS) and polyisoprene (PI) polymers in the feed.

FIG. 4 is an illustration (schematic) of a Janus particle at an oil-water interface. The contact line is pinned, so that the energy to displace the particle from the surface is less sensitive to changes in solvent polarities.

FIG. 5 is an illustration (schematic) of a Janus particle (at left) with a hydrophilic face formed from a high Tg polymer and an oleophilic (hydrophobic) face formed from a low Tg polymer. The oleophilic face contacts and adsorbs an oil layer from skin. The Janus particle is kept dispersed by the hydrophilic face. In contrast a polymer micelle (at right) has a hydrophilic steric block layer (shown as lines on the outside) that prevents contact with the oil layer and, thereby, minimizes uptake into the hydrophobic micelle core (shown as an inner disk).

FIG. 6 is an illustration (schematic) of a geometric Janus micelle. Controlled assembly of Janus particles creates a structure (geometric Janus micelle) with interstitial regions into which oil can wet and be sequestered.

FIGS. 7A-7D show the morphology of homopolymer and Janus particles. FIG. 7A shows a transmission electron microscope (TEM) image of a nanoparticle formed of polylactic acid (PLA) homopolymer (Mw=12,300 g/mol). FIG. 7B shows a TEM image of a nanoparticle formed of polycaprolactone (PCL) homopolymer (Mw=14,000 g/mol). FIGS. 7C and 7D show TEM images of Janus nanoparticles formed of PLA homopolymer and PCL homopolymer.

FIG. 8A shows the size distribution of PLA homopolymer nanoparticles. FIG. 8B shows the size distribution of PCL homopolymer nanoparticles. FIG. 8C shows the size distribution of Janus nanoparticles (JN) formed of PLA homopolymer and PCL homopolymer.

FIG. 9 shows TEM images of Janus nanoparticles formed of PLA homopolymer and polyethylene oxide-block-polycaprolactone (PEO-b-PCL) block copolymer (BCP) (PEO block Mw=5,000 g/mol and PCL block Mw=13,500 g/mol).

FIG. 10 shows photographs of oil (hexane)-in-water emulsions stabilized by PLA nanoparticles, PCL nanoparticles, and PLA/PCL Janus nanoparticles at room temperature over time (0 hr. (at formation), 1 hr. (post formation), 2 hr, 8 hr, and 24 hr.).

FIG. 11 shows photographs of oil (hexane)-in-water emulsions stabilized by mass equivalent PEO-b-PCL block copolymer, number equivalent PEO-b-PCL block copolymer, and PLA/PEO-b-PCL Janus nanoparticles at 23° C. over time (0 hr. (at formation), 1 hr. (post formation), 2 hr, 8 hr, and 24 hr.).

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

Published international applications WO2015/200054 (filed Jun. 16, 2015; published Dec. 30, 2015) and WO2015/130835 (filed Feb. 25, 2015; published Sep. 3, 2015) are hereby incorporated by reference in their entirety.

The terms “particle”, “nanoparticle”, “colloid”, and “nanocolloid” are used interchangeably herein, unless another meaning is indicated by the context. The term “Janus” refers to a particle having two distinct surfaces, for example, having two surfaces of different polymers. The term “Janus” can also refer to a characteristic of such a particle or group of particles, such as “Janus morphology” or “Janus phase”.

A “polymer” can be considered to be formed of repeating chemical units (“monomers”). A “homopolymer” is generally formed of only a single type of chemical unit (the same monomer) (although there can be variations in stereochemistry, e.g., an isotactic polymer has substituents located on the same “side” of the macromolecular backbone, a syndiotactic polymer has substituents located at alternating “sides” along the macromolecular backbone, and an atactic polymer has substituents located at random “sides” along the macromolecular backbone). A copolymer can be formed of two or more chemical units (different monomers). A “random copolymer” has the different monomers randomly placed along the polymer chain. A “block copolymer” has one monomer grouped sequentially along the chain, the other monomer grouped sequentially at a different location along the chain (and so forth, if there are more than two chemically distinct monomers). A block copolymer can be a “diblock” copolymer, having a sequence of a first monomer on one end coupled to a sequence of a second monomer on the other end. A block copolymer can be a “triblock” copolymer, having a sequence of a first monomer on one end, coupled to a sequence of a second monomer in the middle of the polymer chain, which is in turn coupled to a sequence of a third monomers on the other end of the polymer chain (the first and the third monomers can be identical to yield an “A-B-A” triblock, or they can be different to yield an “A-B-C” triblock). Higher order “multi-block” copolymers can be formed. Unless otherwise specified, a “polymer” can refer to an entire homopolymer or to a block or segment of a copolymer. For example, “a surface including polyethylene oxide” may refer to a surface that includes a polyethylene oxide homopolymer, or to a surface that includes a polyethylene oxide block or segment of a polyethylene oxide-polycaprolactam block copolymer (PEO-b-PCL.

Methods according to the invention apply to a broad range of polymer chemistries and cost effective processes to produce Janus particles. Processes according to the invention can produce bi- or tri-phasic, polymeric Janus particles which have distinct polymer chemistry in the phases and which have distinct surface chemistries on the faces.

Dissimilar polymers can be combined to create single colloids with phase-separated surfaces (Walther, A. et al., Janus discs, J. Am. Chem. Soc., 2007, 129, 6187-98). This can be accomplished by incorporating two or more distinct polymers into a single polymer chain to create block co-polymers that are then induced to assemble into Janus particles through a series of surface-based processing steps. However, multi-processing steps on a two-dimensional surface makes scalability non-trivial. Furthermore, particle size is fixed by the molecular weight (Mw) of the co-polymer (Walther, A. et al., Janus discs, J. Am. Chem. Soc., 2007, 129, 6187-98; Pochan, D. J. et al., Multicompartment and multigeometry nanoparticle assembly, Soft Matter 2011, 7, 2500).

The solution-based self-assembly of homopolymer molecules into nanoscale objects allows for the creation of Janus colloids with structural and compositional complexity (Jang, S. G. et al., Striped, ellipsoidal particles by controlled assembly of diblock copolymers, J. Am. Chem. Soc. 2013, 135, 6649-57; Yamashita, N. et al., Preparation of hemispherical particles by cleavage of micrometer-sized, spherical poly(methyl methacrylate)/polystyrene composite particle with Janus structure: effect of molecular weight, Colloid Polym. Sci. 2013, 292, 733-738; Higuchi, T. et al., Spontaneous formation of polymer nanoparticles with inner micro-phase separation structures, Soft Matter 2008, 4, 1302-1305; Kiyono, Y. et al., Preparation and Structural Investigation of PMMA-Polystyrene “Janus Beads” by Rapid Evaporation of an Ethyl Acetate Aqueous Emulsion, e-Journal Surf. Sci. Nanotechnol. 2012, 10, 360-366). Such particles can be fabricated by dissolving multiple, chemically distinct polymers in a mutually favorable solvent and gradually altering the solubility character of the solution until the polymer molecules co-precipitate into self-organized structures. The final morphology adopted by the colloids via solution self-assembly can be unique to the particular processing conditions used, so that the range of architectures accessible to any one method is limited. This limitation on the range of accessible architectures constrains the applicability of these previous approaches. Also, the slow precipitation steps result in uncontrolled size distributions of the resulting particles. This is a major problem with the slow precipitation approaches, since control of particle size is essential in applications of these structured nanoparticles. The use of small amphiphilic surfactant molecules or polymeric stabilizers in the solution volume or a collection solution in which the polymers co-precipitate can mask the compositional heterogeneity and interfacial properties of the particle surface. The existing solution-based approaches usually operate under batch conditions with residence times of days or hours (Jang, S. G. et al., Striped, ellipsoidal particles by controlled assembly of diblock copolymers, J. Am. Chem. Soc. 2013, 135, 6649-57; Wang, Y. et al., Colloids with valence and specific directional bonding, Nature, 2012, 491, 51-5). For example, Higuchi and coworkers demonstrated the use of a slow solvent evaporation technique to form imperfect, micro-phase-separated nanoparticles from a solution containing polyisoprene and polystyrene. (Higuchi, T. et al., Spontaneous formation of polymer nanoparticles with inner micro-phase separation structures, Soft Matter, 2008, 4(6), 1302-1305). They were able to find, amongst the large array of other structures in the particles produced by the slow solvent evaporation, Janus particles. However, the Higuchi synthesis process had the following disadvantages: (1) it could not control the size of the assembled particle; (2) the slow precipitation could not produce nanoparticles with controlled stoichiometry (i.e., the least soluble polymer would precipitate first and produce nanoparticles that do not necessarily contain both polymers in a controlled ratio, so the process is not generalizable to arbitrary polymer pairs); and (3) the process took two days to slowly evaporate solvent from a 200 mL beaker, so that it was not scalable.

Therefore, a challenge remains to develop a continuous, scalable, and simple particle fabrication system that offers comprehensive control over multiple particle features such as particle size, surface domain size, and surface topology (Jang, S. G. et al., Striped, ellipsoidal particles by controlled assembly of diblock copolymers, J. Am. Chem. Soc. 2013, 135, 6649-57; Higuchi, T. et al., Spontaneous formation of polymer nanoparticles with inner micro-phase separation structures, Soft Matter 2008, 4, 1302-1305). Current strategies to produce polymer-polymer and/or polymer-inorganic Janus particles, including surface coating by vapor deposition, coating via Pickering emulsion, layer-by-layer self-assembly, biphasic electrified jetting, surface initiated polymerization, polymerization in microfluidic devices, and polymer phase separation, are not pathways to scalable technologies in which kilograms/day of material can be produced continuously.

A successful and scalable approach has two requirements: (1) a process must produce nano or microparticles of essentially uniform size; and (2) the polymeric contents of the nano or microparticle must spontaneously phase separate during the formation process to form a bi-, tri-, or multi-phasic structure.

The phase separation of polymer blends, a self-directed physical process capable of generating multi-domain structures at the nanoscale, can be used to fabricate structured multi-face particles (Sai, H. et al., Hierarchical porous polymer scaffolds from block copolymers, Science 2013, 341, 530-4). The complex structures associated with polymer phase separation may be transferred to colloids in a controllable manner by confining the volume and time scale in which polymer de-mixing takes place. The phase separation of dissimilar polymers precipitated from a common solvent via a confined impinging jet mixer can be induced through FNP. Polymer de-mixing is driven to occur within precipitating nanodroplets of polymer and solvent as the solvent rapidly exchanges on the order of milliseconds with a non-solvent during micro-mixing (Johnson, B. et al., Mechanism for Rapid Self-Assembly of Block Copolymer Nanoparticles, Phys. Rev. Lett. 2003, 91, 118302). The process unit, FNP, has advantages that render it a transformative route to Janus nanocolloids, including the following: i) a one-step and continuous process; ii) a room temperature and low energy process; and iii) proven scalability greater than 1400 kg/day of colloids.

With FNP, precursor polymers can be uniformly dispersed in a single phase solution and aggregate into particles with sufficiently uniform size. The FNP method can provide simultaneous control over particle size, surface functionality, and compositional anisotropy as the assembly process is scaled in the production of particles, such as Janus colloids assembled from two simple homopolymers. Tuning the molecular weight of the homopolymers and increasing the number of polymer components in the system can facilitate the formation of multi-faced and multi-lobal nanocolloids, respectively. Incompatible polymers with different properties can be self-assembled into nanocolloids with controllable surface topology by simultaneously reducing the timescale and solution volume over which they undergo self-assembly. FNP can create polymeric Janus particles with multi-phasic bulk and surface properties.

Alternatively, an emulsion of sufficiently uniform size can be created by mechanical dispersion. The emulsion comprises an internal dispersed phase containing the polymer components dissolved in a common solvent to afford a single phase fluid, and an external phase fluid in which the solvent phase is not completely miscible. The emulsion is stable under formation conditions. The solvent phase is then removed by an evaporation or extraction process, so that the polymers spontaneously phase separate during that removal or stripping process. This can create polymeric Janus particles with multi-phasic bulk and surface properties.

The nano or microparticles can be created without additional stabilizers. In that case the final Janus particle is in its final form. However, it may be necessary or desirable to process the particles with an added amphiphilic stabilizer to increase the stability of the particle or enable production at higher dispersed phase concentrations. In cases where a stabilizer is added it can be substantially removed from the surface by a subsequent step to unmask the particle, so that the two Janus (or three or more multi-face) surfaces display different surface chemistries. Examples of stabilizers that can be used include sulfonated alkyl surfactants, sodium dodecyl sulfate, ethoxylated sulfonate surfactants, cationic surfactants, amine oxide surfactants, zwitterionic surfactants, amphoteric surfactants, ethylene oxide surfactants based on alkyl ethers, ethylene oxide surfactants based on nonylphenols, surfactants based on sorbitan oleates, surfactants based on sugars such as glucose-based surfactants, polymeric surfactants such as polyethylene oxide-co-polybutylene oxide surfactants, such as the Pluronic or Pluroximer surfactants from BASF, polymeric stabilizers based on polyvinyl caprolactam and polycaprolactone, polymeric stabilizers based on partially hydrolyzed polyvinyl alcohol, polymeric stabilizers based on polyethylene oxide, natural products polymeric stabilizers based on substituted cellulose, such as hydroxypropyl cellulose, natural products polymeric stabilizers based on hydrophobically modified starches, lipids, and lecithin.

Without being bound by theory, the stability of a purely hydrophobic Janus particle may arise from the strong negative charge arising from hydroxyl adsorption (Beattie, J. K. et al., The surface of neat water is basic, Faraday discussions, 2009, 141, 31-39). Excessive salt is observed to precipitate the particles, which is consistent with this view.

Molecular weight ranges of polymers useful for forming Janus particles range from the lowest molecular weight that creates macroscopic phase separation, for example, 800 Da (Dalton or g/mol) for polystyrene molecules in a blend with polyisoprene, up to 10⁵ Da molecular weight or up to 10⁷ Da molecular weight. For examples, polymers having molecular weights ranging from about 800 Da, 1 kDa, 3 kDa, 5 kDa, 8 kDa, 10 kDa, 12 kDa, 15 kDa, 20 kDa, 30 kDa, 100 kDa, 300 kDa, 1000 kDa, or 3000 kDa, to about 1 kDa, 3 kDa, 5 kDa, 8 kDa, 10 kDa, 12 kDa, 15 kDa, 20 kDa, 30 kDa, 100 kDa, 300 kDa, 1000 kDa, 3000 kDa, or 10,000 kDa can be used. Macroscopic, bulk phase separation can be determined by any classical experimental technique, including imaging the final Janus particle.

The Janus formation process is widely applicable, and is not limited to specific polymer chemistry. The following polymers with purely hydrophobic terminal units, with hydroxyl (OH), and with carboxyl (COOH) units have been formed into Janus particles. Janus particles have been formed from polymers over the molecular weight range 9,000 to 1,000,000 Da. Examples of Janus particles formed are provided in Tables 1-3.

TABLE 1

| | | MW | End |
|---|---|---|---|
| Polymer | Name | (kg/mol) | functionalization |
| 1 | Atactic Polystyrene | 16.5 | None (hydrogen |
| | | | terminated) |
| 2 | Atactic Polystyrene | 1500 | None (hydrogen |
| | | | terminated) |
| 3 | Carboxy terminated | 16.5 | carboxylic acid |
| | Polystyrene | | |
| 4 | hydroxy-terminated | 16 | hydroxyl group |
| | Polystyrene | | |
| 5 | Polyisoprene (1,4 | 11 | None (hydrogen |
| | addition) | | terminated) |
| 6 | Polyisoprene (1,4 | 1000 | None (hydrogen |
| | addition) | | terminated) |
| 7 | Polybutadiene | 9.1 | None (hydrogen |
| | | | terminated) |
| 8 | Polybutadiene(1,4 | 18 | None (hydrogen |
| | addition) | | terminated) |
| 9 | Hydroxy-terminated | 12.5 | hydroxyl group |
| | Polybutadiene | | |
| 10 | Polyvinylcyclohexane | 25 | None (hydrogen |
| | | | terminated) |

TABLE 2
Janus Particle Polymer Pairings (Bicomponent)
1 & 5
2 & 6
4 & 5
4 & 9
3 & 5
3 & 9
10 & 8

TABLE 3
Janus Particle Polymer Groupings (Tricomponent)
10, 8, 1
10, 7, 1

Because Janus structures can be made from polymers with a wide range of terminal functionality, the Janus particles can be reacted after formation to impart desirable surface properties on the Janus faces. For example, the COOH or OH can be reacted with amine groups or the OH with acid chlorides to attach more hydrophilic entities on one Janus face. This can enhance the hydrophobicity/hydrophilicity difference between the two faces and can enhance the interfacial stabilization properties of the construct. A range of other surface modifications are possible, and they can be designed to impart a variety of Janus properties.

A hydrophilic polymer is generally attracted to water molecules (or attracts water molecules), and can exhibit phenomena such as water on its surface having a low contact angle, water spreading on its surface, swelling in the presence of water, and/or being soluble in water (although it need not necessarily be water soluble). A hydrophobic polymer is generally not attracted to water molecules (it may appear to be repelled from water molecules or appear to repel water molecules), and can exhibit phenomena such as water on its surface having a high contact angle, water beading on its surface, lack of swelling in the presence of water, and/or being insoluble in water. Most hydrophobic polymers are lipophilic or oleophilic, and can exhibit phenomena such as swelling in the presence of fats, oils, and low-polarity and/or non-polar solvents and/or being soluble in fats, oils, and low-polarity and/or non-polar solvents (although certain polymers, such as certain silicones and fluorocarbons, may be hydrophobic, but not lipophilic). The hydrophilicity or hydrophobicity of a polymer can be understood in a relative sense. That is, although a first polymer may be more hydrophilic than a second polymer, the second polymer may be more hydrophilic than a third polymer. Thus, that second polymer may be relatively hydrophobic when compared with the first polymer, but that second polymer may be relatively hydrophilic when compared with the third polymer. Therefore, a Janus particle having the first polymer on a face A and the second polymer on a face B may be considered to have the face B (with the second polymer) as the “hydrophobic face,” whereas a Janus particle having the second polymer on a face A and the third polymer on a face B may be considered to have the face A (with the second polymer) as the “hydrophilic face.”

Colloidal particles can be used to stabilize emulsions. Emulsions stabilized with colloidal particles can be termed Pickering emulsions (Binks, B. P. et al. Langmuir 2007, 23 (7), 3626-3636. Binks, B. P. & Lumsdon, S. O. Langmuir 2001, 17 (15), 4540-4547.). Solid particles of intermediate wettability may exhibit higher surface activity than surfactants and can irreversibly adsorb to the oil-water interface (Binks, B. P. Curr. Opin. Colloid Interface Sci. 2002, 7 (1-2), 21-41.). Emulsions prepared with such particles may exhibit a high resistance to droplet coalescence and retain their emulsified properties longer than emulsions having small molecule or no stabilizing agents (Chevalier, Y. & Bolzinger, M. A. Colloids Surfaces A Physicochem. Eng. Asp. 2013, 439, 23-34.). Surfactants do not create an irreversibly adsorbed interfacial boundary like solid particles, but instead exist in dynamic equilibrium with the oil and water phases, continuously adsorbing and desorbing on short time scales (Aveyard, R. et al. Adv. Colloid Interface Sci. 2003, 100-102 (SUPPL.), 503-546.). The different interfacial behaviors of surfactants and solid particles influence the coalescence, phase-transfer, and rheological properties of the emulsions of which they are a part (Binks 2002. Tambe, D E. & Sharma, M. M. Adv. Colloid Interface Sci. 1994, 52 (C), 1-63. Tcholakova, S. et al. Phys. Chem. Chem. Phys. 2008, 10 (12), 1608-1627.). Emulsions stabilized with colloidal particles can find applications in industries such as the pharmaceutical, healthcare, cosmetic, and personal care industries, as well as have other industrial applications.

The surface activity of particles at the interface is dependent, inter alia, on the contact angle, θ, that particles make with the oil-water interface (Aveyard 2003). θ quantifies the wettability of particles and is a determinant of the preferred emulsion type, either oil-in-water or water-in-oil. Interfacial tension at the oil-water interface, γOW, is another influential parameter of surface activity. The effect of these parameters on emulsion stability are described by the energy, E, needed to remove a particle from the oil-water interface, given by:

E=πR²γ_{OW}(1±cos θ)² (1)

where R is the radius of the sphere, yow is the interfacial tension, and θ is the contact angle measured in the aqueous phase (Binks, B. P. & Lumsdon, S. O. Langmuir 2000, 16, 8622-8631.). Unlike the behavior of E for surfactants, E falls rapidly on either side of 0=90° for solid particles of intermediate wettability, such that for a particle fully immersed in either the oil or the aqueous phase, the energy of detachment is comparable to its thermal energy, kT (Binks 2000). A highly hydrophobic or hydrophilic particle, therefore, desorbs easily from an oil-water interface with room temperature thermal fluctuations and provides little utility as a stabilizing agent. A particle with intermediate wettability, however, provides more stabilization.

Unlike surfactants, which have both hydrophobic and hydrophilic regions, homogenous particles are only partially wetted by either phase, owing to their uniform composition (Binks 2002). Surfactants can be mixed with surface active particles (Binks, B. P. et al. Langmuir 2007b, 23 (3), 1098-1106.). However, the stabilization efficiency of such systems depends on processing parameters and on both particle and surfactant concentration, as high amounts of either component can lead to droplet coalescence (Binks 2007b).

Amphiphilic, two-faced Janus particles (Deng, R. et al. Macromolecules 2016, 49, 1362-1368. Ye, X. et al. ACS Nano 2016.) can be used for stabilizing emulsions. A Janus particle has two physically distinct regions within or on the surface of the particle. Polymer Janus nanoparticles can include two different polymer regions. For example, a Janus particle can have an asymmetric structure, in which the surface of one side of the particle has one component, e.g., a first polymer, and the surface of the other side of the particle has a different component, e.g., a second polymer. That is, Janus particles can have a “two-faced” form.

The asymmetric structure of Janus particles allows for the simultaneous tailoring of chemical surface properties and wettability in order to allow the particle to act as a macroscopic surfactant with the interfacial stabilizing ability of a colloid. Thus, Janus particles can have combined emulsifying capabilities of a surfactant and a colloidal particle. Asymmetric Janus particles can inhibit the coalescence of viscous polymer droplets in an immiscible polymer blend at temperatures above ambient conditions (Bryson, K. C. et al. Macromolecules 2015, 48(12), 4220-4227.). Janus particles may be useful as emulsion stabilizers (Binks, B. P. & Fletcher, P. D. I. Langmuir 2001b, 17 (16), 4708-4710. Glaser, N. et al. Langmuir 2006, 22 (12), 5227-5229.). This can be quantified by calculation of the total surface free energy for a Janus particle at the interface, given by:

$${E}_{A} ( β ) = 2 π {R}^{2} [ {γ}_{A O} ( 1 + cos α ) + {γ}_{PO} ( cos β - cos α ) + {γ}_{P W} ( 1 + cos α ) - \frac{1}{2} {γ}_{OW} ( {sin}^{2} β ) ] \qquad ( 2 )$$

for β≤α, where A, P, W, and O indicate the apolar, polar, water, and oil regions of the Janus particle (Kumar, A. et al. Soft Matter 2013, 9 (29), 6604-7202.). However, biocompatible emulsion stabilizers have not been previously developed.

PLA and PCL are biodegradable polymers. They exhibit good biocompatibility, are easily processed, and have degradation products that are removed through physiological metabolic pathways (Thomson, R. C. et al. J. Biomater. Sci. Polym. Ed. 1995, 7 (1), 23-38.). For example, PLA and PCL can be used in therapeutic applications, such as drug delivery vehicles, scaffolds for cell transplantation, and stents for the treatment of narrowed arteries (Sahoo, S. K. et al. Biomacromolecules 2005, 6, 1132-1139. Windecker, S. et al. Lancet 2008, 372 (9644), 1163-1173.).

A fully biodegradable Janus particle made from polylactic acid (PLA) and polyethylene oxide-block-polycaprolactone (PEO-b-PCL) that effectively stabilizes oil-in-water emulsions for long periods of time and that was made through flash nanoprecipitation (FNP) is described in this present application (Johnson, B. K. & Prud'homme, R. K. Australian Journal of Chemistry; 2003, 56, 1021-1024. Sosa, C. et al. Macromolecules 2016, 49, 3580-3583.). The biodegradable composition of these particles, as well as their surface activity and the amphiphilic properties that arise from their Janus structure, makes them well-suited for and may address the biocompatibility, environmental, and toxicity challenges posed by traditional methods of emulsion stabilization. In the following, the morphology of a Janus nanocolloid made from the biodegradable polymers PLA and PEO-b-PCL through the FNP process and its effectiveness as a stabilizer of oil-in-water emulsions is described. The FNP process is continuous and scalable, and is a one-step, continuous process for the production of Janus nanocolloids.

In addition to stabilizing oil-in-water emulsions, PLA/PEO-b-PCL Janus nanoparticles (nanocolloids) may be useful in technological applications such as drug delivery and medical imaging.

### Flash NanoPrecipitation

Flash NanoPrecipitation (FNP) can be used for the production of organic and organic/inorganic nanoparticles. The mean particle diameter of these nanoparticles can be in the range of from 30 to 2000 nm, for example, from about 50 to 800 nm. For example, the mean particle diameter of these nanoparticles can be from about 10, 20, 30, 50, 60, 100, 200, 300, 400, 500, 600, 700, 800, 1000, 1200, 1500, 2000, 4000, 5000, 6000, or 10,000 nm to about 20, 30, 50, 60, 100, 200, 300, 400, 500, 600, 700, 800, 1000, 1200, 1500, 2000, 4000, 5000, 6000, 10,000, or 20,000 nm. FNP can form particles of narrow size distribution. For example, of the nanoparticles formed, at least 90% can have a diameter less than 800 nm, and at most 10% can have a diameter less than 50 nm. For example, of the nanoparticles formed, at least 90% can have a diameter less than 50,000, 20,000, 10,000, 6000, 5000, 4000, 2000, 1500, 1000, 800, 500, 400, 300, 200, 100, 60, 50, 30, 20, or 10 nm, and at most 10% can have a diameter less than 20,000, 10,000, 6000, 5000, 4000, 2000, 1500, 1000, 800, 500, 400, 300, 200, 100, 60, 50, 30, 20, 10, or 5 nm.

The FNP process uses micromixing geometries to mix an incoming, solvent stream in which a polymer is dissolved (so that it can also be termed a polymer solution stream) with a non-solvent stream to produce supersaturation levels as high as 10,000 with mixing times of about 1.5 ms. The solvent stream can be miscible with the non-solvent stream. For example, supersaturation levels can range from about 100, 300, 1000, 3000, 10,000, 30,000, 100,000, or 300,000 to about 300, 1000, 3000, 10,000, 30,000, 100,000, 300,000, or 1,000,000, and mixing times can range from about 0.01, 0.03, 0.1, 0.3, 1, 1.5, 3, 10, 15, 30, 100, or 300 ms to about 0.03, 0.1, 0.3, 1, 1.5, 3, 10, 15, 30, 100, 300, or 1000 ms. It is desirable that these mixing times are shorter than the nucleation and growth times of nanoparticle assembly, so that the size of the nanoparticles formed is constrained. The solvent stream and non-solvent stream can be further mixed with a collection solution, for example, a collection solution that includes a stabilizer such as an amphiphilic surfactant molecule. Nanoparticles can be formed for a variety of pharmaceutical compound, imaging agent, security ink, and drug targeting applications (Johnson, B. K. et al., Chemical processing and micromixing in confined impinging jets, AIChE J. September 2003, 49(9), 2264-2282; Johnson B. K. et al., Mechanism for rapid self-assembly of block copolymer nanoparticles, Phys. Rev. Lett. Sep. 12, 2003, 91(11); Johnson, B. K. et al. Flash NanoPrecipitation of organic actives and block copolymers using a confined impinging jets mixer, Australian J. Chem. 2003, 56(10), 1021-1024; Johnson, B. K. et al., Nanoprecipitation of organic actives using mixing and block copolymer stabilization, Abstracts of Papers of the American Chemical Society September 2003, 226, U487-U487; Johnson B. K. et al., Engineering the direct precipitation of stabilized organic and block copolymer nanoparticles as unique composites, Abstracts of Papers of the American Chemical Society September 2003, 226, U527-U527; Johnson, B. K. et al., Nanoprecipitation of pharmaceuticals using mixing and block copolymer stabilization, Polymeric Drug Delivery II: Polymeric Matrices and Drug Particle Engineering 2006, 924, 278-291; Ansell, S. M. et al., Modulating the therapeutic activity of nanoparticle delivered paclitaxel by manipulating the hydrophobicity of prodrug conjugates, J. Med. Chem. June 2008, 51(11), 3288-3296; Gindy, M. E. et al. Preparation of Poly(ethylene glycol) Protected Nanoparticles with Variable Bioconjugate Ligand Density, Biomacromolecules October 2008, 9(10), 2705-2711; Gindy, M. E. et al., Composite block copolymer stabilized nanoparticles: Simultaneous encapsulation of organic actives and inorganic nanostructures, Langmuir January 2008, 24(1), 83-90; Akbulut M. et al., Generic Method of Preparing Multifunctional Fluorescent Nanoparticles Using Flash NanoPrecipitation, Adv. Funct. Mater. 2009, 19, 1-8; Budijono, S. J. et al., Exploration of Nanoparticle Block Copolymer Surface Coverage on Nanoparticles, Colloids and Surfaces A—Physicochemical and Engineering Aspects, 2010; Budijono, S. J. et al., Synthesis of Stable Block-Copolymer-Protected NaYF₄:Yb³⁺, Er³⁺ Up-Converting Phosphor Nanoparticles, Chem. Mat. 2010, 22(2), 311-318; D'Addio, S. M. et al., Novel Method for Concentrating and Drying Polymeric Nanoparticles: Hydrogen Bonding Coacervate Precipitation, Molecular Pharmaceutics March-April 2010, 7(2), 557-564; Kumar, V. et al., Fluorescent Polymeric Nanoparticles: Aggregation and Phase Behavior of Pyrene and Amphotericin B Molecules in Nanoparticle Cores, Small December 2010, 6(24), 2907-2914; Kumar, V. et al., Stabilization of the Nitric Oxide (NO) Prodrugs and Anticancer Leads, PABA/NO and Double JS-K, through Incorporation into PEG-Protected Nanoparticles, Molecular Pharmaceutics January-February 2010, 7(1), 291-298; D'Addio, S. M. et al., Controlling drug nanoparticle formation by rapid precipitation, Adv. Drug Delivery Rev. May 2011, 63(6), 417-426; Kumar, V. et al., Fluorescent Polymeric Nanoparticles: Aggregation and Phase Behavior of Pyrene and Amphotericin B Molecules in Nanoparticle Cores, Small December 2011, 6(24), 2907-2914; Shan, J. N. et al., Pegylated Composite Nanoparticles Containing Upconverting Phosphors and meso-Tetraphenyl porphine (TPP) for Photodynamic Therapy, Adv. Functional Materials July 2011, 21(13), 2488-2495; Shen, H. et al., Self-assembling process of flash nanoprecipitation in a multi-inlet vortex mixer to produce drug-loaded polymeric nanoparticles, J. Nanoparticle Res. September 2011, 13(9), 4109-4120; Zhang, S. Y. et al., Photocrosslinking the polystyrene core of block-copolymer nanoparticles, Polym. Chem. March 2011, 2(3), 665-671; Zhang, S. Y. et al., Block Copolymer Nanoparticles as Nanobeads for the Polymerase Chain Reaction, Nano Lett. April 2011, 11(4), 1723-1726; D'Addio, S. M. et al., Constant size, variable density aerosol particles by ultrasonic spray freeze drying, Intl J. Pharmaceutics May 2012, 427(2), 185-191; D'Addio, S. M. et al., Effects of block copolymer properties on nanocarrier protection from in vivo clearance, J. Controlled Release August 2012, 162(1), 208-217; D'Addio, S. M. et al., Optimization of cell receptor-specific targeting through multivalent surface decoration of polymeric nanocarriers, J. Controlled Release May 2013, 168(1), 41-49; Figueroa, C. E. et al., Effervescent redispersion of lyophilized polymeric nanoparticles, Therapeutic Delivery 2013, 4(2), 177-190; Figueroa, C. E. et al., Highly loaded nanoparticulate formulation of progesterone for emergency traumatic brain injury treatment, Therapeutic Delivery 2013, 3(11), 1269-1279; Pinkerton, N. M. et al., Formation of Stable Nanocarriers by in Situ Ion Pairing during Block-Copolymer-Directed Rapid Precipitation, Mol. Pharmaceutics 2013, 10, 319-328; Pinkerton, N. M. et al., Gelation Chemistries for the Encapsulation of Nanoparticles in Composite Gel Microparticles for Lung Imaging and Drug Delivery, Biomacromolecules 2013; DOI: 10.1021/bm4015232). Flash NanoPrecipitation can be used with stabilizing block copolymers to produce nanoparticles. Alternatively, FNP can be used for the production of polystyrene particles without an added stabilizer or amphiphilic copolymer. Nanoparticles over the size range of 60 to 200 nm with polydispersities comparable to those produced by emulsion polymerization were obtained using only electrostatic stabilization.

A polymer and/or copolymer can be dissolved to form the polymer solution process stream at a concentration in a range of from about 0.1 wt %, 0.2 wt %, 0.5 wt %, 1 wt %, 2 wt %, 5 wt %, 10 wt %, or 20 wt % to about 0.2 wt %, 0.5 wt %, 1 wt %, 2 wt %, 5 wt %, 10 wt %, 20 wt %, or 40 wt %. A person of skill in the art will appreciate that a factor such as the economics of a process can constrain a lower bound of concentration, and that factors such as the viscosity of the process solution or the solubility limit of the copolymer in the polymer solution process stream can constrain an upper bound of concentration. For example, if the viscosity of the polymer solution process stream is much greater than that of the non-solvent stream, mixing of the first process solution with the non-solvent stream may be inhibited. A person of skill in the art will appreciate that factors such as the molecular weight of the copolymer and the composition of the copolymer can affect the maximum concentration that can be attained in the polymer solution before the viscosity becomes too high.

FNP overcomes the limitations of previous approaches that did not control the size of the assembled nanoparticles, were unable to produce nanoparticles with controlled stoichiometry, and were slow and not scalable. With FNP, nanoparticle size can be controlled. Rapid micromixing to a uniform high supersaturation produces diffusion limited aggregation, and the aggregating solutes or polymers “stick” randomly to each other, so that each particle contains the stoichiometric ratio of solutes that are introduced into the FNP micromixer. Although the process is random, because each nanoparticle contains polymer chains on the order of 50,000 Da molecular weight, the variance in concentration between particles is small. The FNP process takes on the order of 15 ms for particle formation. The FNP process is continuous and scalable. The FNP process has been scaled to 1400 kg/day by BASF.

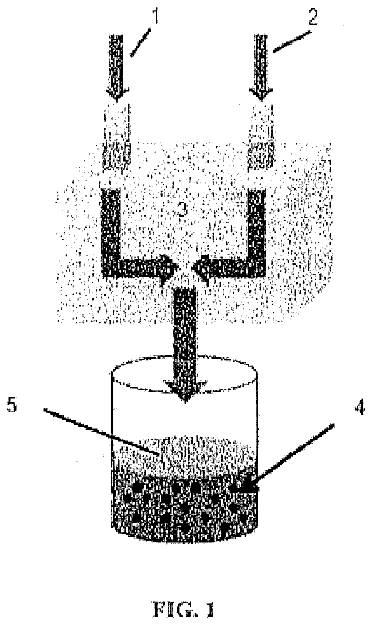

Thus, FNP is a room temperature, low energy, one-step, rapid, and continuous route to produce polymer-polymer Janus nanoparticles. A schematic of the FNP process is illustrated in FIG. 1. The mixing occurs in a central cavity 3 fed by two incoming streams 1 and 2 that are high velocity linear jets of fluid. The one stream 1 contains the polymers dissolved in a solvent. The other stream 2 is of a non-solvent for the polymer. The compositions and ratios of the streams are chosen so that after mixing in the central cavity 3, the polymers are no longer dissolved and rapid precipitation occurs (Johnson, B. K. et al., AIChE J 2003, 49, 2264; Johnson, B. K. et al, Phys. Rev. Lett. 2003, 91; Johnson, B. K. et al., Aust. J. Chem. 2003, 56, 1021; Pustulka, K. M. et al., Mol. Pharmaceutics, 2013, 10, 4367). The nanoparticles formed 4 can be collected in a collection solution 5. Different mixing geometries can be used in this process, as long as the selected mixing geometry selected produces rapid micromixing to control precipitation (Burke, P. A. et al., International Patent Application PCT/US2011/031540 and U.S. Published Patent Application US20130037977). For example, a confined impingement jet (CIJ) mixing system can be used to combine a solvent stream containing fully dissolved polymers with a stream that is a non-solvent for the polymers to drive polymer phase-separation in a confined nano-droplet assembly volume. The polymer solution rapidly mixes with the non-solvent for a few milliseconds to induce self-assembly of the polymers into kinetically frozen nanoparticles. When used to form polymeric Janus particles two polymers may be dissolved in the solvent (e.g., an organic solvent) of stream 1, a polymer solution stream. However, other hydrophobic components such as small molecule drugs, imaging agents, particles, and therapeutic agents can be successfully encapsulated into polymeric nanoparticles by FNP (Shan, J. et al, Adv. Funct. Mater. 2011, 21, 2488.; Kumar, V. et al., Small 2010, 6, 2907; Pinkerton, N. M. et al., Biomacromolecules 2014, 15, 252).

A wide range of solvents and non-solvents that are miscible can be used in the process. Solvents include materials in which the polymer components are soluble. For example, the solvent is miscible with the non-solvent. Nonsolvents include materials in which the polymer components are not soluble or are only sparingly soluble. For example, the solvent can be a non-aqueous solvent, such as an organic solvent or a low polarity solvent, and the non-solvent can be water, a predominantly aqueous phase, or a high polarity solvent. Alternatively, the solvent can be water or a high polarity solvent (for example, if the polymer to be dissolved is a hydrophilic polymer) and the non-solvent can be a non-aqueous solvent or a low polarity solvent. Alternatively, the solvent and the non-solvent can be selected from two different non-aqueous solvents. The solvent or the non-solvent can be polar or nonpolar (or have an intermediate polarity) and can be protic or aprotic. Examples of materials that can be used as solvents or non-solvents include water, alcohols, such as methanol, ethanol, isopropanol (2-propanol), and n-propanol (1-propanol), carboxylic acids, such as formic acid, acetic acid, propanoic acid (propionic acid), butyric acid, furans, such as tetrahydrofuran (THF), dioxane, 1,4-dioxane, furfuryl alcohol, ketones, such as acetone and methyl ethyl ketone (MEK), other water-miscible solvents, such as acetaldehyde, ethylene glycol, propanediol, propylene glycol (propane-1,2-diol), 1,3-propanediol, butanediol, 1,2-butanediol, 1,3-butanediol, 1,4-butanediol, pentanediol, 1,5-pentanediol, 2-butoxyethanol, glycerol, triethylene glycol, dimethyl sulfoxide (DMSO), ethylamine, diethanolamine, diethylenetriamine, methyl diethanolamine, dimethylformamide (DMF), and pyridine, acetonitrile, methyl isocyanide, esters, such as methyl acetate and ethyl acetate, ethers, such as diethyl ether and dimethoxyethane, carbon disulfide, halogenated organics, such as carbon tetrachloride, alkanes, such as heptane, alkenes, such as hexene, cycloalkanes, such as cyclohexane, aromatic hydrocarbons, such as toluene, other organic and inorganic materials, and mixtures of these. Additional non-liquid compounds that aid in the solvent quality of the streams may be added and are also considered part of the solvent. For example, a surfactant, a salt, or a cosolvent may be added to a solvent and considered part of the solvent. These excipient compounds may or may not be in the final nanoparticle or microparticle construct, depending on the requirements of the final product. A further description of solvent compositions useful for processing by FNP has been presented in B. K. Johnson, R. K. Prud'homme, US Patent Application Pub. US 2012/0171254 A1, Jul. 5, 2012.

FNP is useful in producing homogenous nanoparticles of various polymers including polystyrene (PS), polymethylmethacrylate (PMMA), polycaprolactone (PCL), and polyethylene oxide (PEO) with controlled diameters and narrow polydispersity indexes (PDIs) (Kumar V. et al., Preparation of lipid nanoparticles: European Patent EP2558074, (2013)). Neither premade nanoparticles nor immobilization steps are required for the FNP process. By simply adjusting the initial polymer concentrations, it is possible to tune the anisotropy of the Janus nanoparticles. Hybrid polymer-inorganic Janus nanoparticles can be made by the FNP process. FNP has been previously described in the following patent documents, which are incorporated by reference into this submission in their entirety:

Preparation of Lipid Nanoparticles, M. Gindy, et al., US Patent Publication, US20130037977 A1, PCT/US2011/031540, publication date Feb. 14, 2013;
A high-loading nanoparticle-based formulation for water-insoluble steroids, C. Figureroa et al., Patent Publication, WO2013063279 A1, PCT/US2012/061945, publication date May 2, 2013;
Particulate constructs for release of active agents, L. D. Mayer et al., Patent Publication US20130336915 A1, Publication date Dec. 19, 2013; and
Process and Apparatuses for Preparing Nanoparticle Compositions with Amphiphilic Copolymers and Their Use, B. K. Johnson et al., US Patent Application Pub., US 2012/0171254 A1, Jul. 5, 2012.
The production of single component polymer nanoparticles by FNP has been described in Zhang, C. et al., Flash nanoprecipitation of polystyrene nanoparticles, Soft Matter 2012, 8(1), 86-93, which is also incorporated herein by reference in its entirety.

The FNP process requires adequate micromixing, which has been described in the patents above. FNP requires that the polymers or inorganic colloids of interest be mutually soluble in a common organic process solvent which is miscible with the non-solvent stream. Water or an aqueous solution can be used as the non-solvent stream and a water-miscible organic solvent can be used as the process solvent stream (e.g., to form the polymer solution stream). With the polymer additives the convergence of the two streams produces a dispersed Janus nanoparticle dispersion in the mixed solvent phase.

The FNP process may be run without a stabilizer additive, so that the process solvent contains the polymers and/or colloids of interest without an amphiphilic stabilizer. Alternatively, amphiphilic stabilizers may be added to either the process solvent phase or the non-solvent phase. It is also possible to reverse the solvent polarity and to precipitate water soluble Janus particles in a non-aqueous non-solvent phase.

Particles may be produced by the FNP process to have, for example, diameters between 10 nm and 4000 nm, between 20 nm and 1000 nm, or between 50 nm and 800 nm. The sizes are the intensity weighted average size determined by dynamic light scattering. Such measurements can be conducted in a Malvern Nanosizer dynamic light scattering (DLS) instrument. The size reported by dynamic light scattering is the intensity weighted diameter, which is used herein to report sizes of the particles produced by the Flash NanoPrecipitation process. The breadth of distribution of the particle diameters can be characterized by values such as the Di90, the intensity-weighted diameter where 90% of the particles have a lesser diameter, the Di50, the intensity-weighted diameter where 50% of the particles have a lesser diameter, and the Di10, the intensity-weighted diameter where 10% of the particles have a lesser diameter. For example, to define a minimum narrowness of distribution of particle diameters, it can be specified that at least 90% of the particles have a diameter less than a nominal Di90 value and that at most 10% of the nanoparticles formed have a diameter less than a nominal Di10 value, or that 80% of the nanoparticles have a diameter greater than or equal to the nominal Di10 value and less than the nominal Di90 value. Alternatively, the Span can be defined as the difference between the Di90 and Di10 values divided by the Di50 value, that is, Span=(Di90−Di10)/Di50. A smaller Span indicates a more narrow distribution of particle sizes, with a Span of zero indicating a monodisperse distribution (i.e., all particles have the same size. The Di10, Di50, and Di90 values are determined from the intensity weighted distribution that is obtained from the dynamic light scattering measurement. These values can be calculated on a mass-weighted basis using standard conversions from intensity- to mass-weighted distributions.

Nanoparticles formed by FNP can include a stabilizing polymer, such as a copolymer of a more polar block coupled with a more nonpolar (less polar) block. The term “block” may be interpreted as either a distinct domain with a single molecular composition, or it may mean a region of the polymer chain which has regions that are predominantly more polar and other regions that are less polar. The polarity may be imparted by the monomers comprising the polymer backbone or grafted pendant groups or chains attached to the main polymer backbone. For example, the copolymer may be amphiphilic (the more nonpolar block is not water soluble), however, this is not a requirement and copolymers may be fully water soluble or fully non-water soluble, as long as solubilities of the blocks differ significantly enough in the nonprocess solvent. The copolymer should self-assemble in the nonprocess solvent, with the more polar blocks precipitating and the more nonpolar blocks remaining soluble. When used in the FNP process to make particles, the more polar blocks go to the core of the particle, and the more nonpolar blocks form a sterically protective shell. The sterically protective shell prevents particle aggregation and prevents percolation of encapsulated material during post processing steps.

Nanoparticles formed by the disclosed process can be formed with graft, block, or random copolymers. For example, these copolymers can have a molecular weight between about 1000 g/mol and about 1,000,000 g/mol, or between about 3000 g/mol and about 25,000 g/mol, or at least about 2000 g/mol.

The copolymers are comprised of repeat units or blocks that have different solubility characteristics. Typically, these repeat units are in groups of at least two comprising a block of a given character. Depending on the method of synthesis, these blocks could be of all the same repeat unit or contain different repeat units dispersed throughout the block, but still yielding blocks of the copolymer with polar and more non-polar portions. These blocks can be arranged into a series of two blocks (diblock) or three block (triblock), or more (multiblock), forming the backbone of a block copolymer. In addition, the polymer chain can have chemical moieties covalently attached or grafted to the backbone. Such polymers are graft polymers. Block units making up the copolymer can occur in regular intervals or they can occur randomly making a random copolymer. In addition, grafted side chains can occur at regular intervals along the polymer backbone or randomly making a randomly grafted copolymer. In graft polymers, polar blocks may be grafted on a non-polar polymer. More commonly, non-polar blocks are grafted on a more polar polymer chain. In graft copolymers, the length of a grafted moiety can vary. Preferably, the grafted segments are equivalent to 2 to 22 ethylene units in length. The grafted hydrophobic groups which create at least one less polar region of the copolymer may comprise tocopherol, tocopherol derivatives, lipids, alcohols with carbon numbers from 12 to 40, cholesterols, unsaturated and/or hydrogenated fatty acids, salts, esters or amides thereof, fatty acids mono-, di- or triglycerides, waxes, ceramides, cholesterol derivatives, or combinations. In addition, the grafting of the polymer backbone can be useful to enhance solvation or nanoparticle stabilization properties. The terms polar and non-polar can be understood in a relative sense. For example, the polarity of two blocks in a block copolymer may be sufficiently different, so that the block copolymer is suitable for use in forming nanoparticles using FNP, although the “non-polar” block may be somewhat polar.

The copolymer used in the compositions and methods of the invention may be comprised of blocks of at least two repeat units or with a minimum contour length the equivalent of at least 25 ethylene units. Contour lengths are the linear sum of the polymer backbone, the molecular dimensions of which can be approximated using the Polymer Handbook, 4th Edition, eds. J. Brandrup, E. H. Immergut, and E. A. Grulke, assoc. ed. A. Abe, D. R. Bloch, 1999, New York, John Wiley & Sons, which is hereby incorporated by reference in its entirety.

### Nanoparticle Formation Through Emulsions Formed by Mechanical Dispersion

In the alternative process, an emulsion can be formed by mechanical agitation using, for example, impellers, rotor-stator mixers, porous plate, or micro-structured plate emulsifiers. For example, the use of mechanical disruption in a uniform shear field with control of internal and external viscosity ratios has been described by Bibbette and used by Pinkerton (Pinkerton, N. M. et al., Formation of Stable Nanocarriers by in Situ Ion Pairing during Block-Copolymer-Directed Rapid Precipitation, Mol. Pharmaceutics 2013, 10, 319-328).

Dilution of the polymer by a solvent in the internal phase achieves miscibility of the polymer species. The solvent phase can be removed from emulsion drops by a “stripping” process. Stripping can be achieved by any means. Two processes are direct evaporation and extraction. In direct evaporation the solvent phase has some limited solubility in the external phase and this solvent transfers from the drop interior, through the external phase, to the external atmosphere where it is removed. This evaporation step can be slow and is dependent on the surface area available to remove the solvent from the external phase.

In extraction an additional component is added to the external phase once the emulsion has been fully formed and stabilized. The added component is one that increases the solubility of the internal solvent into the external fluid phase. The increased solubility “strips” the internal solvent from the emulsion drops and transfers it to the external phase.

By either stripping process the increase in polymer concentration inside the emulsion drop creates sufficient polymer:polymer interactions, so that phase separation is achieved and the Janus structure is established. For example, particles can be produced that are between 20 nm and 20,000 nm, between 20 nm and 6000 nm, between 30 nm and 1000 nm, or between 50 nm and 800 nm.

Stabilizers and their Removal

Stabilizers that have been incorporated into either the FNP process or the emulsion by a mechanical dispersion process may need to be substantially removed from the final Janus formulation, depending on the application of the particles.

In a first approach, if the stabilizers have a solubility in the external phase of greater than, for example, 10⁻³ wt %, then they can be removed by solvent exchange and diffusion. This can be done by batch dialysis, by tangential flow ultrafiltration, by centrifugation and decanting, or other processes for removing soluble impurities from particulate suspensions. Once the amphiphilic stabilizer is removed the intrinsic properties of the polymers comprising the Janus core will be exposed.

A second approach involves specific complexation of the surfactant to remove it from the particle surface. One such example is the complexation of surfactants, notably sodium dodecyl sulfate (SDS), using cyclodextrins. The binding constant of SDS to cyclodextrin has a higher affinity than binding to the particle surface. Thus, the surfactant can be removed from the surface. This process has been described for the removal of SDS from hydrophopic associative polymers, and has been used in the synthesis and purification of copolymers using cyclodextrins. The SDS:cyclodextrin complex is stable in the particle dispersion, but it may be desirable to remove the soluble SDS:cyclodextrin complex by one of the methods presented above. SDS is a representative interfacial stabilizer for either FNP or emulsion processing, but other surfactants strongly binding with cyclodextrin can also be used.

In some FNP and emulsion processes it may be desirable to use larger amphiphilic polymers, block copolymers, or surfactants whose solubility is so low that they cannot be removed by the processes described above. For this, a third approach is to use cleavable amphiphilic stabilizers in which the hydrophilic moiety in the stabilizer is attached to the hydrophobic moiety by a linker that may be broken or cleaved. The result is that the hydrophobic moiety is left on the surface and the hydrophilic moiety is dispersed in the external aqueous phase. For an external hydrophobic solvent phase the system is reversed. Cleavable linkages may be esters, orthoesters, ketal, disulfides, or other groups well known in the field that are cleaved by hydrolysis, redox reactions, exchange reactions, enzymatic attack, or other chemical or biochemical reactions that can be initiated by changes in pH, redox conditions, or the addition of catalytic species. In some cases, this third approach using cleavable amphiphilic stabilizers may be desirable to produce nanoparticles that do not contain amphiphilic components in the final formulation, the cleaving process renders the stabilizer no longer amphiphilic.

If the amphiphilic anchoring species is dilute enough on the particle surface, then its presence on the surface will not alter the desirable Janus surface properties.

In another embodiment, when the amphiphilic anchoring block is high enough in molecular weight, for example, above 900 Daltons, it can become part of the Janus phase. For example, an FNP or emulsion process can be conducted with an amphiphilic block copolymer having the hydrophobic block of the same type as the material composing the Janus particle. After Janus particle formation the amphiphilic anchoring block is cleaved removing the soluble portion from the particle surface, while leaving the anchoring block anchored in the polymer matrix. Two or more amphiphilic stabilizers can be used, each with the hydrophobic block being that of one of the polymer phases in the Janus core. The amphiphilic stabilizers will partition on the particle surface, driven by the enthalpic energy of phase separation, which drives the phase separation of the Janus core. Once the soluble components are cleaved the Janus particle will have separate phases which now include the component from the amphiphilic stabilizer. The ratio of amphiphilic stabilizers is adjusted to approximately reflect the volume ratio of the Janus particle core. For example, if the Janus particle is to have a 50:50 ratio of two polymers, the stabilizers should be used in approximately a 50:50 ratio. The exact ratio depends on both the size of the hydrophobic and the hydrophilic blocks to create an optimal surface area ratio.

Without intending to be bound be theory, Flory-Huggins theory can be applied to understand the micro-phase separation in nanoparticle (NP) cores that results in the formation of Janus particles. The Chi parameter, χ, characterizes the strength of interactions between dissimilar polymers and χN parameterizes the total interaction energy of a polymer with N statistical segments. Chi values and the number of monomers per statistical segment are known for most polymers.

Without intending to be bound by theory, the interfacial energy of the two or more polymers can play a role in the Janus structure obtained. From an argument of the total free energy of a Janus particle, it is expected that Janus structure will arise if the absolute value of the surface energy difference between the external (water) phase and polymer A and B is greater than the interfacial energy between components A and B:

γ_{AB}<1.46|(γ_{BW}−γ_{AW})|

If this equality does not hold then a core-shell morphology can be formed. Thus, the formation of a core-shell versus a Janus morphology is an important structural feature that can be controlled.

Janus particles with some fraction of the polymer composition being a block copolymer that has desirable phase behavior properties with the two or three other polymer components can be formed. This enables Janus particle formation by tuning the interfacial energy between the major homopolymer components.

### Isolation of Janus Particles

Post processing steps can be applied to the particle phases to concentrate the Janus particles, remove residual process solvent, change the process solvent, or change the non-solvent. Concentration can be effected by ultrafiltration, selective flocculation, for example, as described by D'Addio, S. M. et al., Novel Method for Concentrating and Drying Polymeric Nanoparticles: Hydrogen Bonding Coacervate Precipitation. Molecular Pharmaceutics March-April 2010, 7(2), 557-564, centrifugation, freeze drying, spray drying, or tray or drum drying. Excipients may be added during the drying or concentrating phases to minimize Janus particle aggregation, or to enhance redispersion. For example, polyethylene glycol from 1000 to 20,000 molecular weight can be used as an excipient.

The ability to create multi-faced nanocolloids, such as Janus particles, from two or more homopolymers not only attests to the versatility of the FNP process, but affords opportunities to construct more sophisticated multi-surface colloids in the future. Through PISA-FNP, colloidal size, anisotropy, and surface functionality can be independently controlled, providing a rapid, solution-based strategy for the formation of soft multi-faced nanocolloids. The simplicity and scalability of the process, furthermore, provides a platform for Janus particle production commensurate with current technological interest.

### Stabilized Homopolymer Particles

A method according to the invention includes making stabilized homopolymer particles. The stabilized homopolymer may have metal formed thereon. The stabilized homopolymer may lack metal formed thereon. The methods of making stabilized homopolymers may include flash nanoprecipitation. According to one embodiment a first fluid stream including a homopolymer (e.g., selected from polystyrene, poly(methyl methacrylate), polycaprolactones, polylactides, polyamides, polysulfones, polyimides, and other polymers) and a solvent therefor is rapidly mixed with a second fluid stream that includes a non-solvent for the homopolymer.

The method of making stabilized homopolymer particles may include merging the mixing streams into a water reservoir that includes a stabilizer (e.g., anionic surfactant) such as sodium dodecyl benzene sulfonate. The homopolymer particles may include only a single polymer. Alternatively, the “homopolymer” particles may also include a small amount of comonomer, in which case they can also be referred to as “near homopolymer” particles. For example, the “homopolymer” or “near homopolymer” may have a small amount (e.g., 5% by weight, or up to about 1%, 2%, 3%, 5%, 10%, or 20% by weight) of acrylic acid comonomer.

The method of making stabilized homopolymer particles may employ a single “homopolymer” (having up to about 5% co-monomer, or up to about 1%, 2%, 3%, 5%, 10%, or 20% by weight co-monomer) and no second polymer in the feed stream. This method enables making homopolymer particles with stabilizers without comonomer, or only a small amount of comonomer.

To summarize some aspects of the invention, a method of forming a multi-faced polymer nanoparticle includes dissolving a first polymer at a first concentration and a second polymer at a second concentration in a solvent to form a polymer solution, selecting a nonsolvent, selecting a mean nanoparticle diameter, selecting the first concentration and second concentration to achieve the selected mean nanoparticle diameter, and continuously mixing the polymer solution with the nonsolvent to flash precipitate the multi-faced polymer nanoparticle in a mixture of the solvent and the nonsolvent. The first polymer can be different from the second polymer. The multi-faced polymer nanoparticle can include a first region, comprising the first polymer at a greater weight fraction than the second polymer, and a second region, comprising the second polymer at a greater weight fraction than the first polymer, with the first region in contact with the second region. In an embodiment, neither the polymer solution nor the nonsolvent include a stabilizer. In a method, the mixing of the polymer solution with the nonsolvent includes mixing with a collection solution. The collection solution can include a stabilizer. The stabilizer can be an amphiphilic surfactant molecule. For example, the stabilizer can be a sulfonated alkyl surfactant, sodium dodecyl sulfate, an ethoxylated sulfonate surfactant, a cationic surfactant, an amine oxide surfactant, a zwitterionic surfactant, an amphoteric surfactant, ethylene oxide surfactant based on an alkyl ether, ethylene oxide surfactant based on a nonylphenol, a surfactant based on sorbitan oleate, glucose-based surfactant, polymeric surfactant, polyethylene oxide-co-polybutylene oxide surfactant, polyvinyl caprolactam based stabilizer, polycaprolactone based stabilizer, polyvinyl alcohol based stabilizer, polyethylene oxide based stabilizer, natural products polymeric stabilizer based on substituted cellulose, hydroxypropyl cellulose, a natural products polymeric stabilizer based on a hydrophobically modified starch, lipid, lecithin, and/or combinations. In an embodiment, the mean particle diameter is in range of from about 30 nm to about 2000 nm, or is in a range of from about 50 nm to about 800 nm. In an embodiment, at least 90% of the nanoparticles formed have a diameter less than 800 nm and at most 10% of the nanoparticles formed have a diameter less than 50 nm. In an embodiment the first region and the second region together include at least 90% of the total volume. For example, the first region and the second region together can include from about 50%, 70%, 80%, 90%, or 95% to about 70%, 80%, 90%, 95%, or 100% of the total volume.

In a method, the first polymer is polystyrene (PS), polyisoprene (PI), polybutadiene (PB), poly(lactic acid) (PLA), poly(vinylpyridine) (PVP), polyvinylcyclohexane (PVCH), poly(methyl methacrylate) (PMMA), polycaprolactone, polyamide, polysulfone, epoxy, epoxy resin, silicone rubber, silicone polymer, and/or polyimide. The second polymer can be polystyrene (PS), polyisoprene (PI), polybutadiene (PB), poly(lactic acid) (PLA), poly(vinylpyridine) (PVP), polyvinylcyclohexane (PVCH), poly(methyl methacrylate), polycaprolactone, polyamide, polysulfone, epoxy, epoxy resin, silicone rubber, silicone polymer, and/or polyimide. The first concentration can be in the range of from about 0.01 to about 30 mg/mL. The second concentration can be in the range of from about 0.01 to about 30 mg/mL. The solvent can be selected from tetrahydrofuran (THF), methyl acetate, ethyl acetate, acetone, methyl ethyl ketone (MEK), dioxane, dimethylformamide (DMF), acetonitrile, methyl pyrrolidone, and dimethyl sulfoxide (DMSO) and/or combinations. The nonsolvent can be selected from the group consisting of water, methanol, ethanol, acetic acid, and/or combinations. In an embodiment, the first polymer is polystyrene (PS), the second polymer is polyisoprene (PI), the solvent is THF, and the nonsolvent is water. In an embodiment, the first polymer is poly(methacrylic acid), the solvent is water, and the nonsolvent is acetone.

In a method, an amphiphilic block copolymer is dissolved in the solvent. The amphiphilic block polymer can include a hydrophobic homopolymer covalently bonded to a hydrophilic homopolymer, with the hydrophobic homopolymer having the same chemical structure as the first polymer. A second amphiphilic block copolymer can be dissolved in the solvent, and the second amphiphilic block polymer can include a second hydrophobic homopolymer covalently bonded to a hydrophilic homopolymer, with the second hydrophobic homopolymer having the same chemical structure as the second polymer.

In a method, the multi-faced polymer nanoparticle is separated from the mixture. For example, the multi-faced polymer nanoparticle can be separated from the mixture by centrifugation, ultrafiltration, and/or spray drying.

In a method, the multi-faced nanoparticle is infused with a medical agent. The medical agent can be a pharmaceutical, an imaging agent, a contrast imaging agent, and/or a radioactive tracer. Alternatively, the multi-faced nanoparticle can be infused with a pesticide or an herbicide.

The first polymer can be a homopolymer or a near homopolymer, the near homopolymer can include a first comonomer and a second comonomer, the first comonomer can be at least 95 wt % of the near homopolymer, and the second comonomer can be at most 5 wt % of the near homopolymer. The mixing of the polymer solution with the nonsolvent can include mixing with a collection solution comprising an anionic surfactant.

An embodiment according to the invention is a group of multi-faced polymer nanoparticles. Each multi-faced polymer nanoparticle can include a first polymer, a second polymer, a first region that includes the first polymer at a greater weight fraction than the second polymer, and a second region that includes the second polymer at a greater weight fraction than the first polymer. The first region can be in contact with the second region. At least 80% of the particles in the group can have a diameter in the range of from about 50 nm to about 800 nm. The first polymer can be a biocompatible polymer.

In a method according to the invention the group of multi-faced polymer nanoparticles is used to strengthen adhesion between a first polymer structure and a second polymer structure at an interface between the first polymer structure and the second polymer structure. The group of multi-faced polymer nanoparticles can be used as an emulsion stabilizer. The group of multi-faced polymer nanoparticles can be used as a foam stabilizer. The group of multi-faced polymer nanoparticles can be used as a foam stabilizer. The group of multi-faced polymer nanoparticles can be used as a solid-liquid interfacial tension modifier.

In an embodiment according to the invention, a multi-faced polymer nanoparticle includes a first polymer and a second polymer, a first region that includes the first polymer at a greater molar fraction than the second polymer, and a second region that includes the second polymer at a greater molar fraction than the first polymer. The first region can be in contact with the second region. The first polymer can be a homopolymer or a near homopolymer, and the near homopolymer can include a first comonomer and a second comonomer. The first comonomer can be at least 95 wt % of the near homopolymer, and the second comonomer can be at most 5 wt % of the near homopolymer.

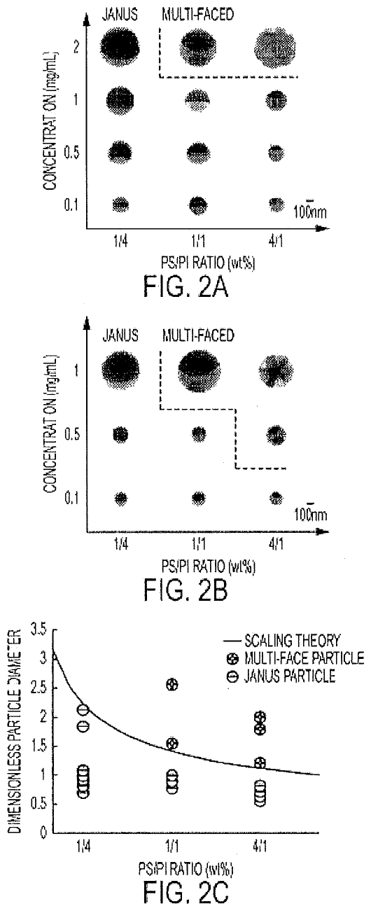

Thus, the phase separation of polymer blends, a self-directed physical process capable of generating multi-domain structures at the nanoscale, can be used to fabricate Janus particles. Flash NanoPrecipitation (FNP) can be used for Precipitation Induced Self Assembly (PISA) to induce the phase separation of dissimilar polymers in nano-domains. This allows for the formation of Janus particles as illustrated in FIGS. 2A-2C. With PISA-FNP key process parameters and molecular features of the polymers can be independently manipulated for the control of particle size, Janus anisotropy, and surface chemistry, features that affect the Janus particle's performance in an application. The PISA-FNP technology is scalable.

### Example 1: Formation of Polystyrene (PS): Polyisoprene (PI) Janus Particle with Amphiphilic Block Copolymer

The FNP process can be used. Along with polystyrene (PS) and polyisoprene (PI) homopolymers, two amphiphilic block copolymers can be added to a tetrahydrofuran (THF) stream. For example, the polymers can be polystyrene-block-polyethylene oxide (PS-b-PEO) (P9669B1-EOS cleavable from Polymer Source, Canada) and a similar PI-b-PEO at a ratio of 50:50 based on the mass of the PEO block. FNP on the mixture can produce nanoparticles that are stable and for which the solvent can be removed by dialysis. To the resulting nanoparticle sample hydrochloric acid (HCl) can be added to produce a pH of 1.5. After 24 hours the sample can be dialyzed against distilled water to obtain a Janus particle dispersion, essentially free of polyethylene oxide (PEO), with a surface chemistry of pure PI and PS.

### Example 2: Polystyrene Polyisoprene Janus Particles

Janus nanocolloids of polystyrene (PS; Mw=16,500 g/mol) and polyisoprene (PI; Mw=11,000 g/mol) (χ_{PS-PI}=0.07) were Formed (Physical Properties of Polymers Handbook, Springer, 2007, 349-355). Tetrahydrofuran (THF) and water were selected as the solvent and non-solvent, respectively. The process conditions employed, e.g., jet velocity ˜1 m/s and a 1 mm orifice, resulted in a Reynolds number ˜3500. Other mixing velocities, for example, in a range from 0.1 m/s to 30 m/s, resulting in other Reynolds numbers, for example, in a range from 300 to 100,000, can be used. For example, mixing velocities ranging from about 0.1 m/s, 0.3 m/s, 1 m/s, 3 m/s, or 10 m/s to about 0.3 m/s, 1 m/s, 3 m/s, 10 m/s, or 30 m/s can be used. For example, Reynolds numbers can range from about 300, 1000, 3000, 10,000, or 30,000 to about 1000, 3000, 10,000, 30,000, or 100,000. Symmetric Janus nanocolloids with a diameter (d) ˜200 nm were formed. To demonstrate the versatility of the process, PS-PI Janus nanocolloids of similar size but varying anisotropy were generated in a systematic manner (see, FIGS. 2A-2C). Simultaneous control over Janus particle size and spatial anisotropy was achieved by simply altering the homopolymer feed ratio and overall feed concentration. This was accomplished without the need for additional process modifications or surfactant interfacial stabilizers. The self-assembled nanocolloids instead acquired their stability from a colloidally-stabilizing surface charge of −33 mV that appears to have resulted from interactions between the surrounding aqueous media and the hydrophobic particle surface (Beattie, J. K. et al., The Surface of Neat Water is Basic, Faraday Discuss. 2009, 141, 31-39). Importantly, the absence of surfactants allows for fully Janus interior and exterior structures, unlike most surfactant-based particle formation processes.

An indispensable feature of the precipitation-induced self-assembly by the FNP (PISA-FNP) method is that key process parameters can be independently manipulated to understand their influence on particle size and morphology as well as gain insight into the mechanism of Janus nanocolloid formation. For instance, representative images of PS-PI Janus nanocolloids processed as a function of overall feed concentration and polymer ratio are shown in FIG. 2A, which provides TEM images of the particles with polystyrene (PS; Mw=16,500 g/mol) and polyisoprene (PI; Mw=11,000 g/mol). FIG. 2A illustrates that increasing the overall (total) feed concentration from 0.1 to 1.0 mg/mL systematically increases the size of the Janus nanocolloids from ˜200 nm to ˜600 nm in diameter. The particle anisotropy can furthermore be tuned independently at each feed concentration by altering the PS-PI polymer ratio from 20:80 to 80:20. The first ratio of components value is the percent of the total polymer mass that is the first polymer, and the second ratio of components value is the percent of the total mass that is the second polymer. Thus, sum of the first ratio and the second ratio is 100. Otherwise stated, the ratio is equal to the ratio of the polystyrene mass concentration to the polyisoprene mass concentration in the stream. As the overall feed concentration is increased to 2 mg/mL, the ability to form Janus nanocolloids depends on the feed ratio of PS to PI. At low PS/PI feed ratios, Janus colloids are observed. However, as the PS/PI feed ratio increases, multi-faced colloids are observed.

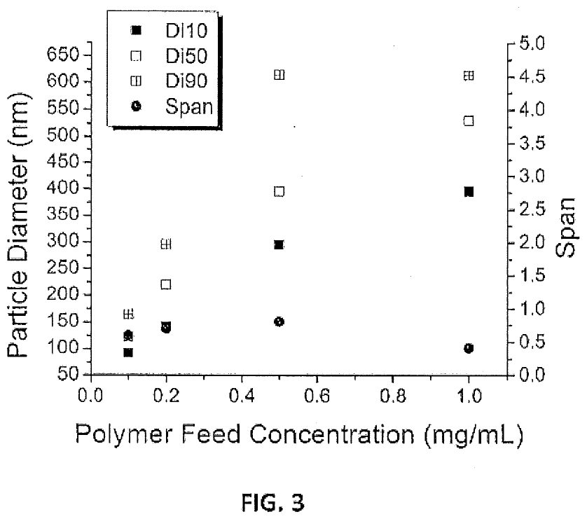

FIG. 3 shows size and polydispersity characteristics of nanoparticles formed for a 50:50 (that is, one-to-one) ratio of polystyrene mass concentration to polyisoprene mass concentration in the feed as a function of total polymer feed concentration. The left-hand axis indicates particle diameter. The Di10, Di50, and Di90 values for the group of particles formed at a given feed concentration are shown by the solid square, hollow square, and crossed square symbols, respectively. As discussed in the context of FIG. 2A, as the total polymer feed concentration increases, the size of the particles formed increases, as defined by each of the Di10, Di50, and Di90 (except that the Di90 value remained constant when increasing total polymer feed concentration from 0.5 to 1.0 mg/mL). The right-hand axis of FIG. 3 indicates the Span (Span=(Di90−Di10)/Di50). The Span increased (indicating a broadening particle size distribution) as the total polymer feed concentration increased from 0.1 to 0.5 mg/mL, but then decreased to its lowest value (indicating the narrowest particle size distribution) as the total polymer feed concentration was increased further to 1.0 mg/mL.

The phase diagram presented in FIG. 2C suggests a competition between the timescales of polymer de-mixing in confined environments and the vitrification time of PS, as set by the volumetric flow rate. According to this hypothesis, manipulating the timescale of either polymer phase separation or solvent exchange can shift the phase boundary between Janus and multi-faced internal structures in a controlled manner.

To investigate this effect, the FNP process was operated under identical conditions, but with an increase in the polymer molecular weight. FIG. 2B shows representative images of PS-PI Janus nanocolloids processed as a function of polymer ratio and overall feed concentration in which the PS and PI Mw were greater, 1,500 kg/mol and 1,000 kg/mol, respectively. At a feed concentration of 0.1 mg/mL, Janus nanocolloids were observed, illustrating that even high Mw polymers have sufficient mobility at dilute concentrations to self-organize into fully segregated polymer domains prior to kinetic trapping. As the overall feed concentration was increased, multi-faced nanocolloids formed, particularly at high PS/PI feed ratios.

Without being bound by theory, the structural features observed in the processed nanocolloids are consistent with the suggestion that internal particle formation proceeds via the phase separation of viscous fluids in a confined environment (see, FIG. 2C). The equilibrium Janus structure adopted by PS and PI at low feed concentrations suggested that the two polymers self-organized into two de-mixed hemispherical domains in order to minimize the total interfacial energy of the ternary phase (polymer-polymer-liquid) system. The Janus morphology, therefore, emerged because the two polymers possessed similar interfacial energies with the THF/water solution (γ_{PS-water}≈Y_{PI-water}) and a low interfacial energy between themselves (Y_{PS-PI}<γ_{PS-water} and Y_{PI-water}), while still forming a stable contact angle. When either the feed concentration or molecular weight of the PS and PI was significantly increased, the timescale over which the polymers phase separated during the assembly process was sufficiently increased above the vitrification time of PS to trap the internal colloidal structure in a non-equilibrium multi-faced state. Since the rate of phase separation decreases by ˜N², where N is the degree of polymerization, the high Mw polymers could generate multi-faced structures at lower polymer feed concentrations than their low Mw counterparts (Bates, F. S., Polymer-Polymer Phase Behavior, Science, 1991, 251, 898-905). The role of PS as a structural trapping agent, moreover, allowed for the enhanced capture of non-equilibrium structures in particles with a high PS content.

The morphology phase diagram for the particles (see, FIG. 2C) is consistent with a simple scaling theory based on surface nucleation (Cogswell, D. A. et al., Theory of Coherent Nucleation in Phase-Separating Nanoparticles, Nano Lett. 2013, 13, 3036-3041). The nearly uniform particle size R(c) for different PS:PI mixtures at the same feed-stream solvent concentration (c) suggests that phase separation occurs mainly after the flash precipitation of homogeneous particles. Spinodal decomposition would lead to random snake-like structures (Balluffi, R. W. et al., Kinetics of Materials, Wiley, 2005) or coherent stripes (Cogswell, D. A. et al., Coherency Strain and the Kinetics of Phase Separation in LiFePO₄ Nanoparticles, ACS Nano 2012, 6, 2215-2225) that are not observed under these process conditions. A potential mechanism is heterogeneous nucleation by surface wetting (Cogswell, D. A. et al., Theory of Coherent Nucleation in Phase-Separating Nanoparticles, Nano Lett. 2013, 13, 3036-3041). While binary solids tend to favor complete coverage of each facet by a single phase (Cogswell, D. A. et al., Theory of Coherent Nucleation in Phase-Separating Nanoparticles, Nano Lett. 2013, 13, 3036-3041), the viscous polymer mixture exhibits partial wetting, so the observed nonequilibrium structures could result from confined capillary instability of the nucleated surface layer, arrested by PS vitrification. Relaxation to the Janus structure occurs if the PS diffusion distance \sqrt{Dτ} during the vitrification time T exceeds the surface layer coalescence distance, scaling as:

$$R ( c ) {( 1 + \frac{P I}{P S} )}^{- 1 / 2} \qquad ( 1 )$$

This dimensionless criterion

$$\tilde{R} = \frac{R}{\sqrt{D τ}} < \sqrt{1 + \frac{P I}{P S}} \qquad ( 2 )$$

successfully predicts (solid line is scaling prediction) the formation of Janus versus patchy particles as shown in FIG. 2C, by collapsing the experimental data from FIGS. 2A and 2B.

Feed concentrations between 0.01 mg/mL and 50 mg/mL can be used for Janus particle formation. For example, feed concentrations from about 0.01, 0.02, 0.05, 0.1, 0.2, 0.5, 1, 2, 5, 10, or 20 mg/mL to about 0.02, 0.05, 0.1, 0.2, 0.5, 1, 2, 5, 10, 20, or 50 mg/mL can be used. The concentration at the upper end is determined by the time required for initial polymer aggregation. Too high of a concentration does not allow uniform particle formation. The lower concentration is determined by the cost of separations of such dilute final products. Concentrations between 0.1 mg/mL and 10 mg/mL are preferred. Polymer ratios between 0.5:99.5 and 99.5:0.5 are possible, depending on the desired application. For example, polymer ratios from about 0.5:99.5, 1:99, 2:98, 5:95, 10:90, 20:80, 30:70, 40:60, 50:50, 60:40, 70:30, 80:20, 90:10, 95:5, 98:2, or 99:1 to about 1:99, 2:98, 5:95, 10:90, 20:80, 30:70, 40:60, 50:50, 60:40, 70:30, 80:20, 90:10, 95:5, 98:2, 99:1, or 99.5:0.5 are possible. The lower and higher limits are determined by the requirement of polymer immiscibility between the two polymers to enable the final Janus particle to have two or more distinct phases. For particles with more than two components the compositions are specified as weight percent ratios, where the sum of the compositions of the components sums to 100.

### Example 3: Janus Particles with Varied Polymer End-Group Functionality and Alternative Polymers

While the surface structure of nanocolloids strongly influences functionality, the material composition of surface domains determines the types of interactions the colloids exhibit with external environments. The PISA-FNP methodology has been extended to two other classes of systems: i) PS-PI Janus nanocolloids with varying polymer end-group functionality; and ii) Janus nanocolloids with new polymer components.

(i) PS-PI Janus nanocolloids were prepared with polymer surfaces containing varying amounts of hydrogen or hydroxyl moieties. This was achieved by using homopolymers with different end-group functionalities in the feed stream, rather than chemically altering the particles post-fabrication. The particles prepared included the following Janus particles: particles prepared with polystyrene and hydroxy-terminated polybutadiene; particles prepared with hydroxy-terminated polystyrene and hydroxy-terminated polybutadiene; and particles prepared with hydroxy-terminated polystyrene and polybutadiene. PS-PI Janus nanocolloids with carboxyl functionalities were also demonstrated. The surface functionality of the Janus colloids can thus be systematically tuned accordingly and allows for further chemical modification of the particles as needed for specific applications.

(ii) The full domain composition was modified to form Janus particles from polystyrene (PS)-poly(lactic acid) (PLA) and polybutadiene (PB)-PLA polymer pairings. The process is therefore capable of producing Janus nanocolloids from varied polymer combinations, including those consisting of biodegradable materials, despite dissimilarity between the properties of paired polymer components.

Janus particles can be formed from polymers selected from other polymers in addition to polystyrene (PS), polyisoprene (PI), polybutadiene (PB), poly(lactic acid) (PLA), poly(vinylpyridine) (PVP), polyvinylcyclohexane (PVCH), poly(methacrylic acid), poly(methyl methacrylate), polycaprolactone, polyamide (e.g., nylon 6,6), polysulfone, epoxy, epoxy resin, silicone polymer, silicone rubber, and polyimide. Polymers used to form Janus particles may be synthetic polymers or natural products. In addition, co-polymers of these polymers may be used, as long as the copolymers phase separate from each other to form multidomain structures. Furthermore, mixtures of polymers may be employed where one or more polymers are miscible into a “first polymer phase”, but the additional polymer(s) that form a “second polymer phase” are immiscible with the first polymer phase. In addition, for multicomponent particles, multiple polymers and mixtures may be used, as long as the polymer phases separate into multiple phases. Many pairs of polymers that are immiscible can be used to form Janus particles. As discussed herein, one or more block copolymers formed from copolymers can be used to form Janus particles. Some additional polymers that can be used to from Janus particles are hydrophobic polymers and hydrophobic polymers formed from moieties such as acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate, cellulose acetate succinate, hydroxypropylmethylcellulose phthalate, and the polymers poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Ilium, L., Davids, S. S. (eds.) Polymers in Controlled Drug Delivery, Wright, Bristol, 1987; Arshady, J. Controlled Release (1991) 17, 1-22; Pitt, Int. J. Phar. (1990) 59, 173-196; Holland, et al., J. Controlled Release (1986) 4, 155-180); hydrophobic peptide-based polymers and copolymers based on poly(L-amino acids) (Lavasanifar, A., et al., Advanced Drug Delivery Reviews (2002) 54, 169-190), poly(ethylene-vinyl acetate) (PEVA) copolymers, silicone rubber, polyethylene, polypropylene, polydienes (e.g., hydrogenated forms of polybutadiene and polyisoprene), maleic anhydride copolymers of vinyl-methylether and other vinyl ethers, polyurethane, polyester urethanes), poly(ether urethanes), poly(ester-urea). For example, preferred polymeric hydrophobes include poly(ethylenevinyl acetate), poly (D,L-lactic acid) oligomers and polymers, poly (L-lactic acid) oligomers and polymers, poly(glycolic acid), copolymers of lactic acid and glycolic acid, poly (valerolactone), polyanhydride, and copolymers of poly(caprolactone) or poly(lactic acid). For non-biologically related applications preferred polymers include polystyrene (PS), polyacrylate, polybutadiene (PB), polysiloxane, polyamide, and polyester.

### Example 4: Janus Particles as Emulsion Stabilizers

Colloidal particles can be used to stabilize emulsions. Emulsions stabilized with colloidal particles can be termed Pickering emulsions (Binks, B. P. et al. Langmuir 2007, 23 (7), 3626-3636. Binks, B. P. & Lumsdon, S. O. Langmuir 2001, 17 (15), 4540-4547. Aveyard, R. et al. Advances in Colloid and Interface Science 2003, 100, 503-546. Binks, B. P. & Clint, J. H. Langmuir 2002, 18, 1270-1273.). Solid particles of intermediate wettability may exhibit higher surface activity than surfactants and can irreversibly adsorb to the oil-water interface (Binks, B. P. Curr. Opin. Colloid Interface Sci. 2002, 7 (1-2), 21-41.).

Emulsions prepared with such colloidal particles may exhibit a high resistance to droplet coalescence and retain their emulsified properties longer than emulsions having small molecules or no stabilizing agents (Chevalier, Y. & Bolzinger, M. A. Colloids Surfaces A Physicochem. Eng. Asp. 2013, 439, 23-34.). Surfactants do not create an irreversibly adsorbed interfacial boundary like solid particles, but instead exist in dynamic equilibrium with the oil and water phases, continuously adsorbing and desorbing on short time scales (Aveyard, R. et al. Adv. Colloid Interface Sci. 2003, 100-102 (SUPPL.), 503-546.). The different interfacial behaviors of surfactants and solid particles influence the coalescence, phase-transfer, and rheological properties of the emulsions of which they are a part (Binks 2002. Tambe, D E. & Sharma, M. M. Adv. Colloid Interface Sci. 1994, 52 (C), 1-63. Tcholakova, S. et al. Phys. Chem. Chem. Phys. 2008, 10 (12), 1608-1627.). Emulsions stabilized with colloidal particles can find applications in industries such as the pharmaceutical, healthcare, cosmetic, and personal care industries, as well as have other industrial applications.

The surface activity of particles at the interface is dependent, inter alia, on the contact angle, θ, that particles make with the oil-water interface (Aveyard 2003). θ quantifies the wettability of particles and is a determinant of the preferred emulsion type, either oil-in-water or water-in-oil. Interfacial tension at the oil-water interface, γOW, is another influential parameter of surface activity. The effect of these parameters on emulsion stability are described by the energy, E, needed to remove a particle from the oil-water interface, given by:

E=πR²γ_{OW}(1±cos θ)² (1)

where R is the radius of the sphere, yow is the interfacial tension, and θ is the contact angle measured in the aqueous phase (Binks, B. P. & Lumsdon, S. O. Langmuir 2000, 16, 8622-8631.). Unlike the behavior of E for surfactants, E falls rapidly on either side of 0=90° for solid particles of intermediate wettability, such that for a particle fully immersed in either the oil or the aqueous phase, the energy of detachment is comparable to its thermal energy, kT (Binks 2000). A highly hydrophobic or hydrophilic particle, therefore, desorbs easily from an oil-water interface with room temperature thermal fluctuations and provides little utility as a stabilizing agent. A particle with intermediate wettability, however, provides more stabilization.

Unlike surfactants, which have both hydrophobic and hydrophilic regions, homogenous particles are only partially wetted by either phase, owing to their uniform composition (Binks 2002). Surfactants can be mixed with surface active particles (Binks, B. P. et al. Langmuir 2007b, 23 (3), 1098-1106.). However, the stabilization efficiency of such systems depends on processing parameters and on both particle and surfactant concentration, as high amounts of either component can lead to droplet coalescence (Binks 2007b).

Amphiphilic, two-faced Janus particles (Deng, R. et al. Macromolecules 2016, 49, 1362-1368. Ye, X. et al. ACS Nano 2016.) can be used for stabilizing emulsions. A Janus particle has two physically distinct regions within and/or on the surface of the particle. Polymer Janus nanoparticles can include two different polymer regions. For example, a Janus particle can have an asymmetric structure, in which the surface of one side of the particle has one component, e.g., a first polymer, and the surface of the other side of the particle has a different component, e.g., a second polymer. That is, Janus particles can have a “two-faced” form.

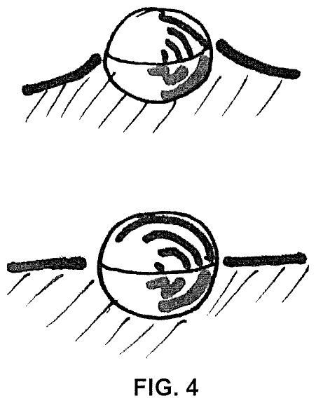

The size scale of a particle can impart high surface free energies. However, to achieve such high surface free energies the contact angle between the oil-water interface and the solid particle surface must be maintained at about 90 degrees (Aveyard 2003). Wetting is sensitive to formulation parameters. An advantage of a Janus particle is illustrated in FIG. 4. Rather than requiring a fixed contact angle, the contact line is pinned as the perimeter of the Janus particle. Janus particles with one hydrophilic face and one hydrophobic face enables emulsion stabilization over a wider range of formulation variables. With the contact line pinned, the energy to displace the particle from the oil-water interface is less sensitive to changes in solvent polarities.

The asymmetric structure of Janus particles allows for the simultaneous tailoring of chemical surface properties and wettability in order to allow the particle to act as a macroscopic surfactant with the interfacial stabilizing ability of a colloid. Thus, Janus particles can have combined emulsifying capabilities of a surfactant and a colloidal particle. Asymmetric Janus particles can inhibit the coalescence of viscous polymer droplets in an immiscible polymer blend at temperatures above ambient conditions (Bryson, K. C. et al. Macromolecules 2015, 48(12), 4220-4227.). Janus particles may be useful as emulsion stabilizers (Binks, B. P. & Fletcher, P. D. I. Langmuir 2001b, 17 (16), 4708-4710. Glaser, N. et al. Langmuir 2006, 22 (12), 5227-5229.). This can be quantified by calculation of the total surface free energy for a Janus particle at the interface, given by:

$${E}_{A} ( β ) = 2 π {R}^{2} [ {γ}_{A O} ( 1 + cos α ) + {γ}_{P O} ( cos β - cos α ) + {γ}_{P W} ( 1 + cos α ) - \frac{1}{2} {γ}_{OW} ( {sin}^{2} β ) ] \qquad ( 2 )$$

for β≤α, where A, P, W, and O indicate the apolar, polar, water, and oil regions of the Janus particle (Kumar, A. et al. Soft Matter 2013, 9 (29), 6604-7202.).

Janus particles (which can be nanoparticles (nanocolloids)) can be formed through the Precipitation Induced Self Assembly—Flash NanoPrecipitation (PISA-FNP) process to have a surface having a hydrophobic face and a hydrophilic face. The hydrophobic face can be obtained by including a purely hydrophobic first polymer in the Janus particle. The hydrophilic face can be obtained by including a second polymer in the Janus particle that has an amine or carboxylic acid functionality at a terminal end, so that the hydrophilic face is charged (for example, the remainder of the second polymer (other than the amine or carboxylic acid functionality at the terminal end, can be hydrophobic or partially or fully hydrophilic). Polyethylene glycol chains can be reacted with the amine groups on the hydrophilic face, for example, using coupling through 1-ethyl-3-(3-dimethylaminopropyl)carbodiimide (EDC) molecules, to provide an electrolyte-independent stabilization of the hydrophilic face of the Janus particles. Minor (small) amounts of amphiphilic block copolymers can be added to the solvent stream in the PISA-FNP process, for example, with the hydrophobic block of the copolymer having the same chemical composition (that is, being formed of the same monomer units) as a homopolymer in the solvent stream. For example, the hydrophobic block of the copolymer can have the same chemical composition as the hydrophobic first polymer. These Janus particles can be designed to optimize their stability against coalescence over time and in centrifugal fields, their formation of emulsions in oil-water systems, and the stability of oil-water emulsions and foams formed with them as a function of temperature and with respect to changes in the polarity of the aqueous and organic phases.

### Example 5: Janus Particles for Surfactant Free Cleansing

Traditional cleaning employs small molecule surfactants. For example, small molecule surfactants are used to solubilize oils and sebum on the skin surface. These small molecule surfactants can interact with skin lipids and proteins and can be irritating to the skin. As described in the below, Janus particles offer a surfactant-free approach to skin cleaning that can avoid such irritation to the skin.

As shown in the left columns of FIGS. 2A and 2B, a Janus particle can be formed with one face including (i.e., presenting to the exterior of the particle) a high glass transition temperature (Tg) polymer (i.e., a rigid solid to form a hard domain) with a hydrophilic surface. The other face of the Janus particle can include (i.e., present to the exterior of the particle) a low Tg oleophilic (and hydrophobic) polymer (i.e., a soft solid in a liquid-like state to form a soft domain). For example, the Janus particles can be composed of polystyrene (PS) (high Tg, Tg of from about 90° C. to about 110° C., for example, 100° C.) functionalized with hydrophilic groups and oleophilic polyisoprene (PI) (low Tg, Tg of from about −90° C. to about −50° C., for example, −70° C.). Other high Tg polymers can be used instead of polystyrene (PS). Other low Tg polymers can be used instead of polyisoprene (PI), for example, polybutadiene (PB) or other rubbery polymers. The production of polystyrene-polyisoprene Janus particles is described in Example 2, above, and images and data for them are presented in FIGS. 2A, 2B, and 3. Example 3 describes the production of Janus particles prepared with hydroxy-terminated polystyrene and polybutadiene (polyisoprene could be substituted for the polybutadiene), and the production of polystyrene-polyisoprene Janus particles (nanocolloids) with carboxyl functionalities.

For example, a high glass transition temperature can be considered to be a glass transition temperature above room temperature (25° C.), and a low glass transition temperature can be considered to be a glass transition temperature below room temperature (25° C.).

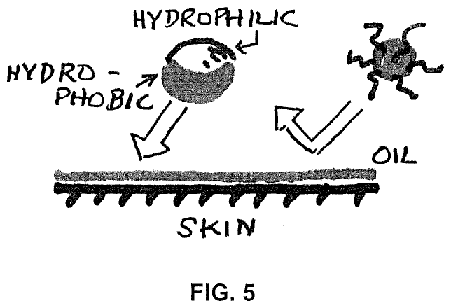

When the low Tg face of the Janus particle comes into mechanical contact with the oil layer, the oil is solubilized through the low Tg oleophilic (hydrophobic) face into the Janus particle; that is, the Janus particle functions as a “nano-eraser”. The hydrophilic functionality on the high Tg face of the Janus particle enables resolubilization of the particles into the rinsing liquid, e.g., water. This is illustrated in the left part of FIG. 5. The unique structure and geometry of the Janus particle is necessary. That is, a purely hydrophobic, low Tg particle would merely adhere and be bound to the oil layer, and would not result in cleansing. If a block copolymer micelle were used, the hydrophilic stabilizing block would protect the hydrophobic micelle core, preventing contact between the oil layer and the imbibing micelle core, as is illustrated in the right part of FIG. 5. That is, the dual (oleophilic and hydrophilic) structure of the Janus nanoparticle provides the dual attributes of an oil “eraser” and a stabilizer. Thus, Janus particles can function as surfactant-free cleansers.

### Example 6. Geometrical Janus Micelles

Controlled aggregation can be used to make geometrical “Janus micelles”.

A conventional surfactant micelle comprises hydrophilic surfactant head groups and hydrophobic surfactant tails. The tails form the condensed core of the micelle, and oils can be solubilized into this hydrophobic micelle core.

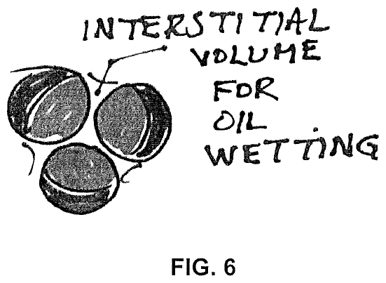

Geometrical Janus micelles can be formed by inducing and controlling the geometric assembly of several Janus particles into structures with a “coordination number”. Because of steric constraints, that is, the individual Janus particles cannot overlap, the Janus micelle cannot collapse or condense, but rather has a core structure with interstitial volume. This is illustrated in FIG. 6, which shows an aggregate of Janus particles, each having a hydrophilic face and a hydrophobic face. For example, the Janus particles can be of the type discussed in Example 5 above, having one face including (i.e., presenting to the exterior of the particle) a high glass transition temperature (Tg) polymer (i.e., rigid solid) with a hydrophilic surface, and having another other face including (i.e., presenting to the exterior of the particle) a low Tg oleophilic (and hydrophobic) polymer (i.e., a soft solid in a liquid-like state). The hydrophobic faces of the Janus particles are oriented (directed) toward the center (the center of mass) of the aggregate. The interstitial volume is between the Janus particles and around the center of the aggregate.

Thus, the uptake of oils is not only through an oleophilic face into an oleophilic region (soft domain) of a Janus particle, but also includes the filling of the interstitial spaces (regions) of the geometrical Janus micelle by capillarity. Oil can wet the oleophilic faces of the Janus particles and fill the interstitial regions by capillarity and be sequestered in these interstitial regions of the geometrical Janus micelle. Thus, higher oil loadings can be achieved with such a geometrical Janus micelle than with the individual Janus particles of which it is formed acting separately.

A geometrical Janus micelle can be formed as follows. Janus particles having a hydrophilic face and an oleophilic face can be formed with the FNP process, for example, as described in Examples 1-5 above. The Janus particles formed can then be suspended or dispersed in a solvent (a process solvent) to form a process solution. For example, the Janus particles can be dispersed in a high polarity solvent, such as water. The process solution can then be continuously mixed with a nonprocess salt solution in a second FNP step to induce the Janus particles to aggregate to form geometrical Janus micelles. That is, the FNP process can be adapted to “salt out” the Janus micelles in an analogous manner as is done for proteins. That is, the assembly of the Janus micelles can be achieved by controlled precipitation of preformed Janus particles during passage through a second FNP process. In the assembly process excess salt or salts are used to precipitate the Janus particles: the hydrophilic faces of Janus particles in an aggregate then point outward, and the hydrophobic (oleophilic) faces of the Janus particles point (orient) inward (toward a center of the aggregate of Janus particles) to create the geometrical Janus micelle.

The sequestering of oils into the geometrical Janus micelles and the cleansing of a surface using the geometrical Janus micelles can be quantified as follows. A model oil can be marked by dissolving into it a lipophilic (hydrophobic) fluorescent dye, such as Nile red dye. The marked model oil then can be coated onto a model surface. The marked model oil coated model surface can then be treated (contacted) with a dispersion or suspension of geometrical Janus micelles, and the surface subsequently rinsed (for example, with water) and the rinse collected. The fluorescence of the rinse containing the geometrical Janus micelles and the fluorescence of the model surface can then be measured and compared with the known quantity of fluorescent dye on the model surface as initially coated with the marked model oil to determine and confirm the quantity of marked oil removed from the model surface by the dispersion or suspension of geometrical Janus micelles.

### Example 7: Formation and Characterization of PLA Nanoparticles

Homopolymer nanoparticles were prepared using polylactic acid (PLA) (Mw=12,300 g/mol, PDI=1.19), purchased from Polymer Source, Inc. The nanoparticles were fabricated through flash nanoprecipitation (FNP) (Sosa 2016). A stream of the polymer dissolved in a good solvent, here, tetrahydrofuran (THF) (purchased from Fisher-Scientific), to form the polymer solution stream was rapidly mixed in the inner chamber of a confined impinging jets (CIJ) mixer with an anti-solvent, here, water (non-solvent stream) (a 0.2 μm filter manufactured by Nanopure Diamond (Barnstead International, Dubuque, Iowa) was used to filter the distilled water used for the experiments). The rapid displacement of the THF by water induced nanoprecipitation of the polymer into nanoparticles, which were quenched in a water bath (aqueous reservoir) and stirred for five minutes. The THF was subsequently removed using a rotary evaporator, resulting in a dispersion of PLA nanoparticles.

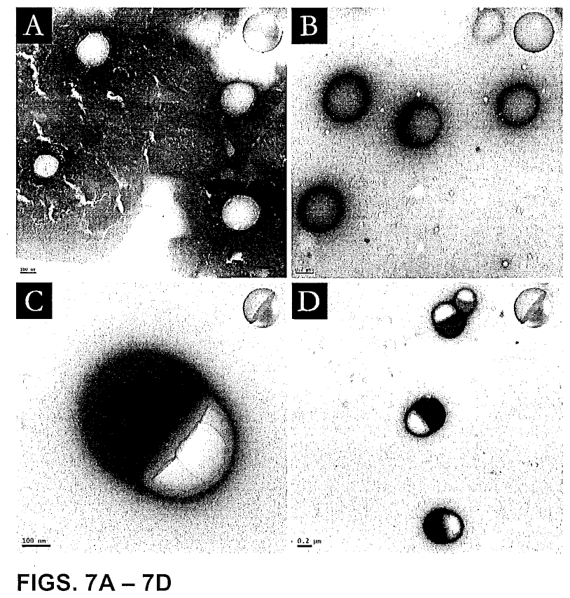

The PLA nanoparticles in the dispersion were imaged by Transmission Electron Microscopy (TEM). The PLA particle dispersion was drop-cast onto a TEM grid and the excess drawn off through the bottom of the grid using a paper tissue (KimWipes, Kimberly-Clark Corp.). A 2% phosphotungstic acid (PTA) solution was drop-cast onto the grid and the excess drawn off through the bottom of the grid using a paper tissue (KimWipes). The PLA sample was then imaged using a regular TEM grid holder on a CM200 FEG-TEM with a Gatan imaging filter. The PLA nanoparticles had a uniform spherical morphology (FIG. 7A).

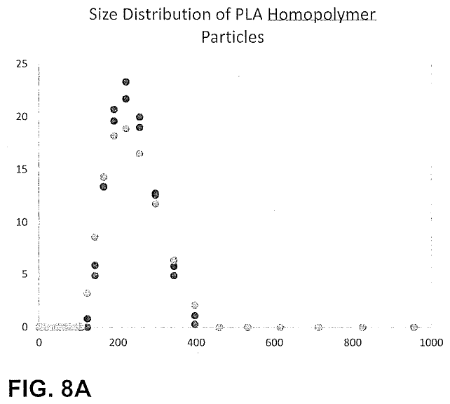

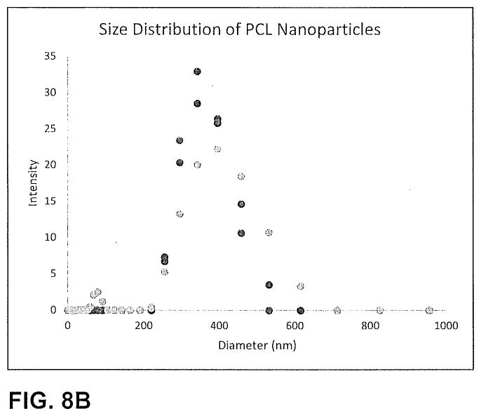

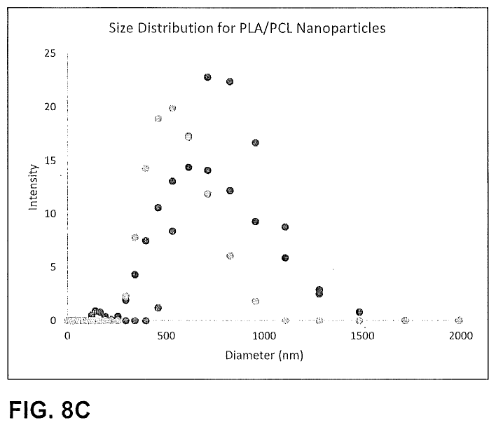

The particle size distribution of the PLA nanoparticles in the dispersion was determined using dynamic light scattering. That is, the diameter of the nanoparticles was measured using a Malvern Zetasizer Nano ZS equipped with a 633 nm wavelength laser and a backscatter detection angle of 73°. The PLA nanoparticles had a mean diameter of 215 nm and had a narrow distribution and were close to monodisperse (FIG. 8A). Thus, near monodisperse dispersions of stable PLA homopolymer nanoparticles were formed through FNP.

### Example 8: Formation and Characterization of PCL Nanoparticles

Homopolymer nanoparticles were prepared using polycaprolactone (PCL) (Mw=14,000 g/mol, PDI=1.4), purchased from Sigma Aldrich. The PCL nanoparticles were fabricated through FNP, using THF as the good solvent and water as the antisolvent, in the same manner as for formation of the PLA homopolymer nanoparticles.

The PCL nanoparticles were prepared for TEM imaging in the same way that the PLA nanoparticles were, but were imaged on the CM200 FEG-TEM with a Gatan imaging filter using a cryogenic TEM grid holder kept at −160° C. with liquid nitrogen for the duration of the imaging. The PCL nanoparticles had a uniform spherical morphology (FIG. 7B).

The particle size distribution of the PCL nanoparticles in the dispersion was determined using dynamic light scattering in the same manner used for the PLA homopolymer nanoparticles. The PCL nanoparticles had a mean diameter of 220 nm and had a narrow distribution and were close to monodisperse (FIG. 8B). Thus, near monodisperse dispersions of stable PCL homopolymer nanoparticles were formed through FNP.

### Example 9: Formation and Characterization of PLA/PCL Nanoparticles

The PLA and PCL polymers were both dissolved in THF as the good solvent. This solution of PLA, PCL, and THF was used as the polymer solution stream for FNP, with water used as the antisolvent, in the same manner as for formation of the PLA homopolymer nanoparticles. The resulting nanoparticles were formed of both PLA and PCL and were Janus (two-faced) anisotropic (asymmetric) nanoparticles.

These PLA/PCL Janus nanoparticles were prepared for TEM imaging by mixing 1 mL (milliliter) of the nanoparticle dispersion with 1 mL of polysorbate 85. This mixture was then drop-cast onto a TEM grid and the excess drawn off through the bottom of the grid using a paper tissue (KimWipes). A 2 mol % PTA solution was then drop-cast onto the grid and the excess drawn off with a paper tissue (KimWipes). The PLA/PCL Janus nanoparticles were then imaged using a regular TEM grid holder on a CM200 FEG-TEM with a Gatan imaging filter. The PLA/PCL nanoparticles had an anisotropic, two-face morphology, as visible in FIGS. 7C and 7D, where the dark region is the PCL domain, stained by the PTA, and the lighter region is the PLA domain.

The particle size distribution of the PLA/PCL nanoparticles in the dispersion was determined using dynamic light scattering in the same manner used for the PLA homopolymer nanoparticles. The PLA/PCL Janus nanoparticles had a mean diameter of 550 nm and had a narrow distribution and were close to monodisperse (FIG. 8C). Thus, stable anisotropic PLA/PCL Janus nanoparticles were formed through FNP.

Polylactic acid (PLA) can be considered to be a high glass transition temperature (Tg) polymer, with a Tg of from about 60° C. to about °65 C. Polycaprolactone (PCL) can be considered to be a low glass transition temperature (Tg) polymer, with a Tg of about −60° C. Both polylactic acid and polycaprolactone are considered to be insoluble in water; however, polylactic acid is generally considered to be more hydrophilic than polycaprolactone.

### Example 10: Formation and Characterization of PLA/PEO-b-PCL Nanoparticles

In pursuit of an improved stabilizing agent, the particle should incorporate not only the anisotropic nature of a Janus particle, but should also exhibit amphiphilicity. Because of the chemical similarity of PLA and PCL, Janus nanoparticles made from these polymers are anisotropic, but do not exhibit true amphiphilicity. The opposing wettability of amphiphilic particles can contribute to an increase in the energy of desorption from an oil-water interface compared to homogenous nanoparticles or Janus nanoparticles with similar wettability on either domain. Thus, following the fabrication of two-component PLA/PCL nanoparticles, PCL was replaced with polyethylene oxide-block-polycaprolactone (PEO-b-PCL) in order to confer amphiphilic character. The PEO-b-PCL (PEO block Mw=5,000 g/mol, PCL block 13,500 g/mol, PDI=1.25) was purchased from Polymer Source, Inc. Otherwise, these PLA/PEO-b-PCL nanoparticles were formed in the same way as the PLA/PCL nanoparticles, that is, the PLA and PEO-b-PCL polymers were both dissolved in THF as the good solvent. This solution of PLA, PEO-b-PCL, and THF was then used as the polymer solution stream for FNP, with water used as the antisolvent, in the same manner as for formation of the PLA/PCL nanoparticles.

In addition to a bipartite morphology, the resulting PLA/PEO-b-PCL nanoparticles also exhibit true amphiphilicity, as the hydrophilic PEO block orients on the surface of the PEO-b-PCL domain, producing a nanoparticle with hemispheres of opposing wettability.

Polyethylene oxide (PEO) can be considered to be a low glass-transition temperature (Tg) polymer, with a Tg of about −67° C. Polyethylene oxide is water soluble and hydrophilic.

In the PLA/PEO-b-PCL nanoparticles, the PLA domain can be considered to be a high glass transition temperature, but relatively hydrophobic domain. The PEO-b-PCL domain can be considered to be a low glass transition temperature, but relatively hydrophilic domain.

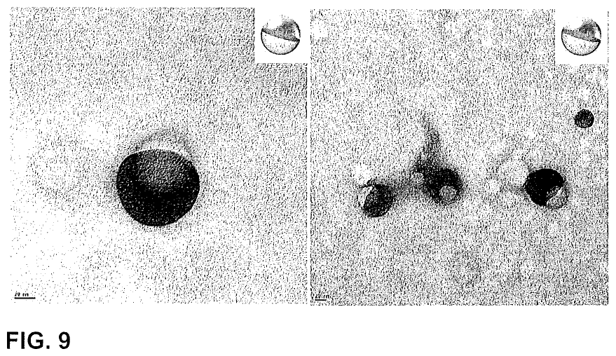

These PLA/PEO-b-PCL Janus nanoparticles were prepared for TEM imaging by mixing 1 mL (milliliter) of the nanoparticle dispersion with 1 mL of polysorbate 85. This mixture was then drop-cast onto a TEM grid and the excess drawn off through the bottom of the grid using a paper tissue (KimWipes). A 2% PTA solution was then drop-cast onto the grid and the excess drawn off with a paper tissue (KimWipes). The PLA/PEO-b-PCL Janus nanoparticles were then imaged using a regular TEM grid holder on a CM200 FEG-TEM with a Gatan imaging filter. The PLA/PEO-b-PCL Janus nanoparticles had an anisotropic, two-face morphology, as visible in FIG. 9. Thus, a PLA/PEO-b-PCL Janus nanoparticle exhibiting both anisotropic and amphiphilic features, composed of fully biodegradable materials, was created.

Other biodegradable and/or biocompatible polymers in addition to or instead of PLA, PCL, and PEO can be used to form biodegradable, anisotropic, and amphiphilic Janus nanoparticles.

### Example 11: Stabilization of Oil-in-Water Emulsions with Nanoparticles

In order to evaluate the effectiveness of the nanoparticles as stabilizing agents, oil-in-water emulsions were prepared.

Emulsions were prepared using an oil (hexane) and a solution of a nanoparticle dispersion of the anisotropic, amphiphilic PLA/PEO-b-PCL Janus nanoparticles, the anisotropic PLA/PCL Janus nanoparticles, the PLA homopolymer nanoparticles, and the PCL homopolymer nanoparticles. For purposes of comparison, emulsions were also prepared using an oil (hexane) and an aqueous solution of PEO-b-PCL block copolymer. The emulsions were made with a 1:1 ratio of hexane to aqueous phase.

Two different PEO-b-PCL concentrations were used for comparison of stabilization by the PEO-b-PCL block copolymer to stabilization by the nanoparticles: a mass equivalent (Me) PEO-b-PCL concentration and a number equivalent (Nₑ) PEO-b-PCL concentration. The mass equivalent PEO-b-PCL solution contained the same number of grams of PEO-b-PCL polymer as the number of grams of PLA and PEO-b-PCL polymers contained in the PLA/PEO-b-PCL Janus nanoparticle. The number equivalent PEO-b-PCL solution was prepared with the underlying assumption that the number of nanoparticles in the PLA/PEO-b-PCL Janus nanoparticle dispersion is equal to the number of PEO-b-PCL entities in the block copolymer solution. That is, the calculation for preparation of number equivalent PEO-b-PCL solution, Nₑ, was carried out as follows.

The volume of a nanoparticle, assumed to be a sphere, is given by

$$V = \frac{4}{3} π {r}^{3}$$

The diameter is known from the DLS measurement and halved to obtain the radius, r. The mass of one nanoparticle is calculated using

mₚ=ρV

where mₚ is the mass of a nanoparticle, p is the density of the nanoparticle (assumed to be I g/cm²), and V is the volume of the nanoparticle. The total number of particles is calculated by

$$totalnanoparticles = \frac{totalpolymermass}{{m}_{p}}$$

It is assumed that the total number of nanoparticles is equivalent to the total number of block copolymer entities. Thus,

totalnanoparticles=totalBCPentities

The mass of PEO-b-PCL block copolymer (BCP) needed to prepare the number equivalent block copolymer solution is then calculated as

$$massBCP = BCPentities \times \frac{1}{{N}_{A}} \times {M}_{W}$$

Where N_{A} is Avogadro's number and M_{W} is the molecular weight of the PEO-b-PCL block copolymer.

Emulsions were prepared by transferring 2 mL of the nanoparticle dispersion or block copolymer solution to a vial and adding 2 mL of hexane (hexane was purchased from Fisher-Scientific). A Vibra-Cell sonicator probe (Sonics & Materials, Inc.) set at 40% amplitude was used to sonicate the sample for 2 minutes.

A sample of the emulsion was taken from the center of the emulsion and dropped onto a glass slide using a glass pipette. A glass coverslip was placed on top of the sample. Droplets were imaged using a microscope and a uEye camera (IDS Imaging Development Systems GmbH, Obersulm, Germany). For emulsion samples that were heated, a Model 280A Isotemp oven (Fischer Scientific) was used.

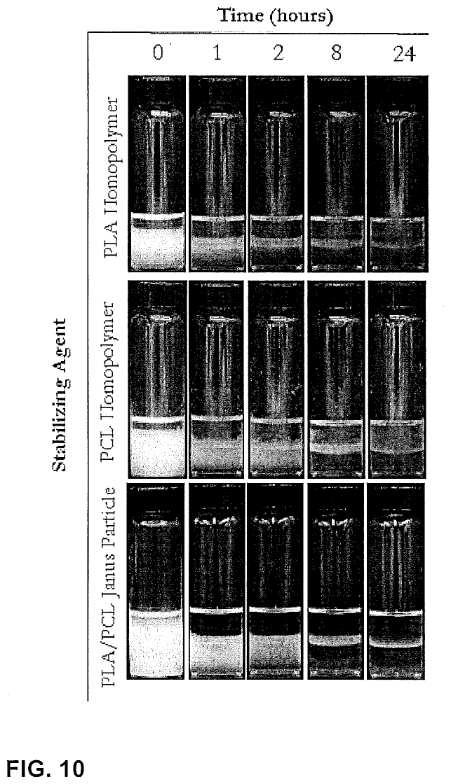

After formation, the emulsions were stored at room temperature and imaged over 24 hours. The stabilizing effectiveness of the nanoparticles and of the block copolymer were compared to each other by visual inspection. As shown in FIG. 10, in the emulsions formed with the PLA homopolymer nanoparticles, PCL homopolymer nanoparticles, and PLA/PCL Janus nanoparticles, after 24 hours, the two phases had nearly completely separated in all three emulsions, suggesting that neither the PLA nor PCL homopolymer nanoparticle dispersions, nor the PLA/PCL nanoparticle dispersion, had performed well as a stabilizing agent.

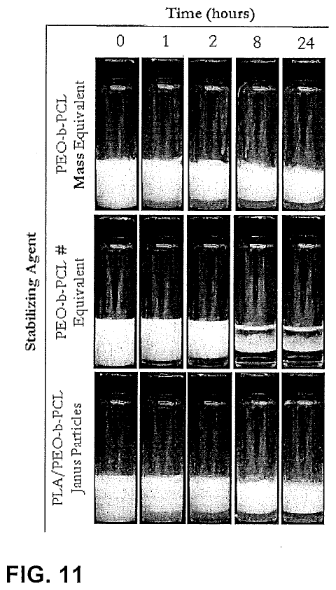

As shown in FIGS. 10 and 11, the anisotropic, amphiphilic PLA/PEO-b-PCL Janus nanoparticles stabilized emulsions to a far greater extent than the PLA, PCL, or PLA/PCL nanoparticles. All emulsions exhibited creaming within the first hour. As shown in FIG. 11, the PLA/PEO-b-PCL Janus nanoparticle stabilized emulsion showed a far lesser extent of destabilization than the Nₑ (#) PEO-b-PCL block copolymer stabilized emulsion and was comparable to the Mₑ PEO-b-PCL block copolymer stabilized emulsion.

In conclusion, the anisotropic, amphiphilic PLA/PEO-b-PCL Janus nanoparticle (nanocolloid), which was made exclusively with biodegradable polymers, is an effective oil-in-water emulsion stabilizing agent. For example, the PLA/PEO-b-PCL Janus nanoparticle can be used to form Pickering emulsions.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

Sharma G. et al., Macromol. Rapid Commun. 2004, 25, 547.
Mei, Y. et al., Langmuir 2005, 21, 12229.
Budijono, S. et al., Nanoparticles for photodynamic therapy: Google Patents; 2009 (PCT/US2008/012485 and US20110022129).
Mayer, L. D. et al., Particulate constructs for release of active agents: Google Patents; 2006 (PCT/US2005/025549).
Mayer, L. D. et al., Particulate constructs for release of active agents: Google Patents; 2007 (EP1786443).
Prud'homme, R. K. et al., Lung targeting dual drug delivery system: Google Patents; 2011 (US20110268803).
York, A. W. et al., Bioactive amphiphilic polymer stabilized nanoparticles with enhanced stability and activity: Google Patents; 2013 (PCT/US2012/050040).

1. An emulsion comprising
an asymmetric Janus (two-faced) nanoparticle comprising a hydrophilic surface as a hydrophilic face and a hydrophobic surface as a hydrophobic face;
a hydrophilic liquid;
a hydrophobic liquid; and
an interface between the hydrophilic liquid and the hydrophobic liquid,
wherein the asymmetric Janus nanoparticle is located at the interface.

2. The emulsion of claim 1, wherein the asymmetric Janus nanoparticle consists of at least one organic polymer.

3. The emulsion of claim 1, wherein the asymmetric Janus nanoparticle comprises a block copolymer comprising a more hydrophilic block and a more hydrophobic block.

4. The emulsion of claim 3, wherein the asymmetric Janus nanoparticle further comprises a homopolymer.

5. The emulsion of claim 4, wherein the block copolymer and the homopolymer are biodegradable and/or biocompatible.

6. The emulsion of claim 1,
wherein the hydrophilic surface comprises a high glass-transition temperature (Tg) polymer and
wherein the hydrophobic surface comprises a low glass-transition temperature (Tg) polymer.

7. The emulsion of claim 6,
wherein the high glass-transition temperature polymer is selected from the group consisting of hydrophilic-functionalized polystyrene (PS), polylactic acid (PLA), and hydrophilic-functionalized polylactic acid (PLA) and
wherein the low glass-transition temperature polymer is selected from the group consisting of polyisoprene (PI) and polybutadiene (PB).

8. The emulsion of claim 1, wherein the hydrophilic surface comprises polymethacrylic acid (PMAA), polyvinylpyridine (PVP), polyethylene oxide (PEO), and/or a hydrophilic functionalized polymer selected from the group consisting of polystyrene (PS), polymethylmethacrylate (PMMA), polylactic acid (PLA), and polyvinylcyclohexane (PVCH), and combinations.

9. The emulsion of claim 1, wherein the hydrophobic surface comprises a polymer selected from the group consisting of polyisoprene (PI), polybutadiene (PB), poly(ethylene-vinylacetate) (PEVA), polycaprolactone (PCL), and combinations.

10. The emulsion of claim 1, wherein the hydrophilic surface comprises a polymer selected from the group consisting of hydroxy-terminated polystyrene (PS), carboxyl-terminated polystyrene (PS), amine-terminated polystyrene (PS), hydroxy-terminated polybutadiene (PB), carboxyl-terminated polybutadiene (PB), amine-terminated polybutadiene (PB), hydroxy-terminated polyisoprene (PI), carboxyl-terminated polyisoprene (PI), amine-terminated polyisoprene (PI), and combinations.

11. The emulsion of claim 1, wherein the hydrophobic surface comprises a polymer selected from the group consisting of polystyrene (PS), polybutadiene (PB), polyisoprene (PI), and combinations.

12. The emulsion of claim 1
wherein the hydrophilic surface comprises polylactic acid (PLA) and
wherein the hydrophobic surface comprises polystyrene (PS), polyisoprene (PI), and/or polybutadiene (PB).

13. The emulsion of claim 1, wherein the asymmetric Janus nanoparticle comprises polylactic acid (PLA) polymer and polyethylene oxide-block-polycaprolactone (PEO-b-PCL) copolymer.

14. The emulsion of claim 1,
wherein the hydrophilic surface comprises polyethylene oxide-block-polycaprolactone (PEO-b-PCL) copolymer and
wherein the hydrophobic surface comprises polylactic acid (PLA) polymer.

15. The emulsion of claim 1, wherein the asymmetric Janus nanoparticle comprises polyethylene oxide-block-polycaprolactone (PEO-b-PCL) copolymer.

16. The emulsion of claim 1, wherein the asymmetric Janus nanoparticle comprises polylactic acid (PLA) polymer and polycaprolactone (PCL) copolymer.

17. The emulsion of claim 13, wherein the polylactic acid (PLA) polymer has a molecular weight in the range of from 10 kDa to 20 kDa.

18. The emulsion of claim 14,
wherein the polyethylene oxide (PEO) component of the polyethylene oxide-block-polycaprolactone (PEO-b-PCL) copolymer has a molecular weight in the range of from 3 kDa to 10 kDa, and
wherein the polycaprolactone (PCL) component of the polyethylene oxide-block-polycaprolactone (PEO-b-PCL) copolymer has a molecular weight in the range of from 10 kDa to 20 kDa.

19. The emulsion of claim 1, wherein the asymmetric Janus nanoparticle has a diameter in the range of from 400 nm to 800 nm.

20. The emulsion of claim 1,
wherein the asymmetric Janus nanocolloid comprises a first hydrophobic polymer, a second polymer, and an amphiphilic block copolymer,
wherein the first hydrophobic polymer forms the hydrophobic face,
wherein the second polymer forms the hydrophilic face,
wherein the second polymer has amine or carboxylic functionality at its terminal end, and
wherein the amphiphilic block copolymer comprises a hydrophobic block formed of the same monomer units of which the first hydrophobic polymer is formed.

21. The emulsion of claim 20, wherein polyethylene glycol polymers are coupled through 1-ethyl-3-(3-dimethylaminopropyl)carbodiimide (EDC) molecules to the amine functionality of the second polymer.

22. A method of cleansing skin or another surface, comprising:
applying a colloid suspension comprising an asymmetric Janus nanoparticle and water (or another liquid) to the skin or other surface; and
rinsing the colloid suspension from the skin or other surface with water (or another liquid),
wherein the asymmetric Janus nanoparticle comprises a high glass transition temperature (Tg) polymer and a low glass transition temperature (Tg) polymer,
wherein the high glass transition temperature polymer has hydrophilic groups at a hydrophilic face of the Janus nanoparticle,
wherein the low glass transition polymer has oleophilic groups at an oleophilic face of the Janus nanoparticle.

23. The method of claim 22,
wherein the high glass transition temperature polymer is polystyrene (PS) functionalized with hydrophilic groups and
wherein the low glass transition temperature polymer is polyisoprene (PI).

24. The method of claim 22,
wherein the high glass transition temperature polymer is polylactic acid (PLA) and wherein the low glass transition temperature polymer is polycaprolactone (PCL).

25. A geometrical Janus micelle, comprising:
an aggregate of a plurality of Janus particles,
wherein each Janus particle has a surface having a hydrophilic face and an oleophilic face and
wherein the oleophilic face of each Janus particle is oriented toward a center of mass of the aggregate.

26. The geometrical Janus micelle of claim 25,
wherein the hydrophilic face comprises a high glass transition temperature polymer and
wherein the hydrophobic face comprises a low glass transition temperature polymer.

27. (canceled)

28. A method of forming the geometrical Janus micelle of claim 25, comprising:
forming the plurality of Janus particles in a first flash nanoprecipitation step;
suspending the plurality of Janus particles in a process solvent to form a process solution; and
continuously mixing the process solution with a nonprocess salt solution in a second flash nanoprecipitation step to form the geometrical Janus micelle,
wherein the geometrical Janus micelle comprises the aggregate of the plurality of Janus particles.
